(19) United States
(12) Patent Application Publication
(10) Pub. No.: US 2023/0023292 A1
(43) Pub. Date: Jan. 26, 2023

(54) LATERAL FLOW IMMUNOASSAY DEVICES AND METHODS OF USING SAME

(71) Applicant: QUANOVATE INC., San Francisco, CA (US)

(72) Inventors: Mengchun QIU, Zhejiang (CN); Xiaodi SUN, Zhejiang (CN); Jijun LIN, Zhejiang (CN); Bin MIAO, Pittsburgh, PA (US); Xing CHANG, San Diego, CA (US); Zheng YANG, Fremont, CA (US)

(21) Appl. No.: 17/936,969

(22) Filed: Sep. 30, 2022

Related U.S. Application Data
(62) Division of application No. 16/777,770, filed on Jan. 30, 2020.
Application No. 16/777,770 is a continuation of application No. PCT/CN2017/095452, filed on Aug. 1, 2017.

(51) Int. Cl. G01N 21/84; G01N 21/47; G01N 21/64; G01N 33/558; G01N 33/68; G01N 33/543

(52) U.S. Cl. CPC G01N 21/8483; G01N 21/474; G01N 21/64; G01N 33/558; G01N 33/6887; G01N 33/689; G01N 33/54388; G01N 2333/78

(57) ABSTRACT

Disclosed herein are devices, systems, methods and kits for performing immunoassay tests on a sample. The immunoassay devices may be used in conjunction with diagnostic reader systems for obtaining a sensitive read-out of the immunoassay results. The immunoassay devices may be especially suited for the detection of at least a first analyte and a second analyte in a sample. The immunoassay devices and methods may utilize a competitive binding-like assay and a sandwich binding assay to detect analytes in a sample.

### CROSS-REFERENCE

This application is a continuation application of International Application Serial No. PCT/CN2017/095452, filed on Aug. 1, 2017, which is incorporated herein by reference in its entirety.

### BACKGROUND OF THE INVENTION

Lateral flow assay devices may provide rapid and cost-effective mechanisms for detecting the presence or absence of analytes in a sample. In some cases, it may be desirable to detect the presence of more than one analyte in a sample. More sensitive methods and devices for performing such tests may be needed. The devices, systems, methods and kits provided herein may be suitable to detect the presence of at least a first and a second analyte in a sample. In some cases, the devices, systems, methods, and kits provided herein may enable a user to perform an immunoassay with more sensitivity than currently available immunoassay devices. In some cases, the devices, systems, methods, and kits provided herein may enable a user to perform a quantitative measurement with higher accuracy and wider dynamic range than currently available immunoassay devices.

### SUMMARY OF THE INVENTION

In one aspect, a method is provided for detecting a presence of at least a first analyte and a second analyte in a biological sample, the method comprising: a) contacting a first end of a test strip with a biological sample suspected of containing the first analyte and the second analyte; b) reacting the biological sample at a labeling zone with a mobilizable first detection reagent conjugated to a first fluorescent label and a mobilizable second detection reagent conjugated to a second fluorescent label, which the first detection reagent specifically binds to the first analyte thereby forming a first analyte-first detection reagent complex and the second detection reagent specifically binds to the second analyte thereby forming a second analyte-second detection reagent complex; c) flowing the biological sample from the labeling zone to a capture zone comprising a first capture region and a second capture region, wherein the first capture region comprises a first capture reagent immobilized thereon which specifically binds to the first detection reagent when the first detection reagent is not in a complex with the first analyte, and wherein the second capture region comprises a second capture reagent immobilized thereon which specifically binds to the second detection reagent-second analyte complex; d) detecting: (i) a first optical signal from the first fluorescent label present at the first capture region which first optical signal decreases with increasing amounts of the first analyte present in the biological sample; and (ii) a second optical signal from the second fluorescent label present at the second capture region which second optical signal increases with increasing amounts of the second analyte present in the biological sample, thereby detecting a presence of the first analyte and the second analyte present in the biological sample. In some cases, the first capture reagent does not displace the first analyte from the first detection reagent if the first analyte is initially bound to first detection reagent prior to step c). In some cases, the first capture reagent does not bind to the first analyte-first detection reagent complex. In some cases, the second capture reagent does not bind to non-complexed second analyte or non-complexed second detection reagent. In some cases, the detecting of d) comprises: (A) illuminating the first capture region and the second capture region with one or more light sources; (B) detecting the first optical signal from the first capture region and the second optical signal from the second capture region with one or more optical detectors; (C) determining an amount of the first analyte present in the biological sample based on a level of the first optical signal; and (D) determining an amount of the second analyte present in the biological sample based on a level of the second optical signal. In some cases, the first capture region is downstream of the second capture region on the test strip. In some cases, the second capture region is downstream of the first capture region on the test strip. In some cases, the first fluorescent label and the second fluorescent label are the same. In some cases, the first analyte is estrone-3-glucuronide (E3G) and the second analyte is luteinizing hormone (LH). In some cases, the first detection reagent comprises an anti-E3G antibody and the second detection reagent comprises an anti-LH antibody. In some cases, the first capture reagent comprises a protein-E3G antigen complex and the second capture reagent comprises an anti-LH antibody. In some cases, a decrease in the first optical signal and an increase in the second optical signal are indicative of a time of an elevated ovulation cycle of a mammal. In some cases, the first fluorescent label and the second fluorescent label are different. In some cases, the first fluorescent label and the second fluorescent label are the same. In some cases, the first detection reagent, the second detection reagent, or both is an antibody or antibody fragment. In some cases, the first detection reagent, the second detection reagent, or both is an antigen or protein-antigen complex. In some cases, the biological sample is blood, urine, or saliva. In some cases, the first capture reagent is an antigen and the second capture reagent is an antibody. In some cases, the method further comprises, after step c) and prior to step d): flowing the biological sample along the test strip from the capture zone to a control zone comprising a first control region and a second control region, wherein the first control region comprises a first control reagent immobilized thereon, which first control reagent binds to the first detection reagent and the second detection reagent, and the second control region comprises a second control reagent immobilized thereon, which the second control reagent binds to the first detection reagent and the second detection reagent. In some cases, the method further comprises detecting optical signals from the first and second fluorescent labels present at the first control region and the second control region. In some cases, the first control reagent and the second control reagent are the same. In some cases, the first control reagent and the second control reagent are different. In some cases, an optical signal at the first control region is greater than an optical signal at the second control region. In some cases, an optical signal detected at the first or second control regions is compared to an optical signal detected at the first or second capture regions. In some cases, the first control reagent, the second control reagent, or both comprise an antibody or antibody fragment. In some cases, both the first control reagent and the second control reagent are anti-mouse IgG antibody.

In another aspect, an assay device is provided for determining a presence of at least a first analyte and a second analyte in a biological sample, the assay device comprising: a test strip defining a flow path and comprising: a) at a first end, a sample zone configured to be contacted with a biological sample suspected of containing the first analyte and the second analyte; b) a labeling zone having absorbed thereon a mobilizable first detection reagent conjugated to a first fluorescent label and a mobilizable second detection reagent conjugated to a second fluorescent label, which the first detection reagent specifically binds to the first analyte thereby forming a first analyte-first detection reagent complex and the second detection reagent specifically binds to the second analyte thereby forming a second analyte-second detection reagent complex; c) a capture zone comprising a first capture region and a second capture region, wherein the first capture region has immobilized thereon a first capture reagent which specifically binds to the first detection reagent when the first detection reagent is not in a complex with the first analyte, and the second capture region has immobilized thereon a second capture reagent which specifically binds to the second analyte-second detection reagent complex, wherein a first optical signal from the first fluorescent label is capable of being detected at the first capture region and which the first optical signal decreases with increasing amounts of the first analyte present in the biological sample, and wherein a second optical signal from the second fluorescent label is capable of being detected at the second capture region and which the second optical signal increases with increasing amounts of the second analyte present in the biological sample. In some cases, the first capture region is downstream of the second capture region on the test strip. In some cases, the second capture region is downstream of the first capture region on the test strip. In some cases, the assay device further comprises, downstream of the capture zone, a control zone comprising a first control region having immobilized thereon a first control reagent which binds to the first detection reagent and the second detection reagent, and a second control region having immobilized thereon a second control reagent which binds to the first detection reagent and the second detection reagent. In some cases, the assay device is configured to be inserted into a reader device for detecting the first and second optical signals from the first and second fluorescent labels. In some cases, the assay device further comprises a readable, or readable and writable chip, configured to be read and wrote by the reader device. In some cases, the readable chip comprises information related to the biological sample, the assay device, or both. In some cases, the first capture reagent does not displace the first analyte from the first detection reagent if the first analyte is bound to the first detection reagent. In some cases, the first capture reagent does not bind to the first analyte-first detection reagent complex. In some cases, the second capture reagent does not bind to non-complexed second analyte or non-complexed second detection reagent. In some cases, the first fluorescent label and the second fluorescent label are the same. In some cases, the first fluorescent label and the second fluorescent label are different. In some cases, the first detection reagent, the second detection reagent, or both is an antibody or antibody fragment. In some cases, the first detection reagent, the second detection reagent, or both is an antigen. In some cases, the biological sample is blood, urine, or saliva. In some cases, the first analyte is estrone-3-glucuronide (E3G) and the second analyte is luteinizing hormone (LH). In some cases, the first detection reagent is an anti-E3G antibody and the second detection reagent is an anti-LH antibody. In some cases, the first capture reagent is a protein-E3G antigen complex and the second capture reagent is an anti-LH antibody. In some cases, a decrease in the first optical signal and an increase in the second optical signal are indicative of a time of an elevated ovulation cycle of a mammal. In some cases, the first control reagent and the second control reagent are anti-mouse IgG antibody.

In another aspect, a diagnostic test system is provided, comprising: a housing, comprising: a) a port for receiving an assay device, the assay device comprising two or more capture regions; b) a reader comprising: i) one or more light sources for illuminating the two or more capture regions; ii) one or more light detectors for detecting optical signals from the two or more capture regions; and c) a data analyzer having one or more processors configured to: A) receive the optical signals; and B) determine an amount of at least a first analyte and a second analyte present in a biological sample based on the optical signals, wherein an optical signal of a first of the two or more capture regions increases with decreasing amounts of the first analyte present in the biological sample, and an optical signal of a second of the two or more capture regions increases with increasing amounts of the second analyte present in the biological sample. In some cases, the diagnostic test system is further configured to detect optical signals from two or more control regions on the assay device. In some cases, the diagnostic test system is further configured to compare optical signals from the two or more control regions with optical signals from the two or more capture regions. In some cases, the assay device comprises any assay device described above. In some cases, the assay device further comprises a second end, configured to be inserted into the port of the diagnostic test system. In some cases, the data analyzer is configured to detect an optical pattern of the optical signals. In some cases, the optical pattern is a binary optical pattern. In some cases, the assay device further comprises a readable chip configured to be detected by the reader. In some cases, the readable chip comprises information related to the biological sample, the assay device, or both. In some cases, the reader is configured to transmit a data output to a mobile device. In some cases, the data output comprises a medical result based on the amount of first analyte and the amount of second analyte present in the biological sample. In some cases, the optical signals are fluorescent signals.

In another aspect, a kit is provided for determining qualitatively or quantitatively the presence of at least a first analyte and a second analyte in a biological sample, the kit comprising: a) an assay device according to any of the above; and b) instructions for using the kit. In some cases, the kit further comprises a diagnostic test system according to any of the above and configured to be in operable communication with the assay device.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts an overview of an immunoassay device in accordance with embodiments of the disclosure. FIG. 1B depicts an exploded top view of an immunoassay device in accordance with embodiments of the disclosure. FIG. 1C depicts an exploded bottom view of an immunoassay device in accordance with embodiments of the disclosure.

FIG. 2 demonstrates application of a fluid sample to an immunoassay device in accordance with embodiments of the disclosure.

FIGS. 3A-3C depict non-limiting examples of immunoassay devices in accordance with embodiments of the disclosure.

FIG. 4 depicts a non-limiting example of an immunoassay device in accordance with embodiments of the disclosure.

FIG. 5 depicts an exploded view of an exemplary diagnostic test system in accordance with embodiments of the disclosure.

FIG. 6 depicts a chamber for receiving and positioning an immunoassay device in accordance with embodiments of the disclosure.

FIG. 7 depicts a diagnostic test system in accordance with embodiments of the disclosure.

FIG. 8 depicts a diagnostic test system in operable communication with an immunoassay device in accordance with embodiments of the disclosure.

FIGS. 9A-B depict a non-limiting example of an optical configuration of a diagnostic test device in accordance with embodiments of the disclosure.

FIGS. 10A-B depict a detector module of a diagnostic test system in accordance with embodiments of the disclosure.

FIGS. 11A-C depict a diagnostic test system in accordance with embodiments of the disclosure.

FIG. 12A depicts a diagnostic test system in operable communication with a mobile device in accordance with embodiments of the disclosure. FIG. 12B depicts a non-limiting example of a mobile device displaying a mobile application in accordance with embodiments of the disclosure.

FIG. 13 depicts a method of using an immunoassay device in accordance with embodiments of the disclosure.

FIGS. 14A-D depict methods of using an immunoassay device in accordance with embodiments of the disclosure.

FIG. 15 depicts a non-limiting example of a labeling reaction performed on an immunoassay device in accordance with embodiments of the disclosure.

FIG. 16 depicts a non-limiting example of a competitive binding-based assay performed on an immunoassay device in accordance with embodiments of the disclosure.

FIG. 17 depicts a non-limiting example of a sandwich-based assay performed on an immunoassay device in accordance with embodiments of the disclosure.

FIGS. 18A-B depict non-limiting examples of a competitive binding-based assay performed on an immunoassay device in accordance with embodiments of the disclosure.

FIGS. 19A-B depict non-limiting examples of a sandwich-based assay performed on an immunoassay device in accordance with embodiments of the disclosure.

FIGS. 20A-B depict non-limiting examples of determining the presence of multiple analytes in a sample using an immunoassay device in accordance with embodiments of the disclosure.

### DETAILED DESCRIPTION OF THE INVENTION

Provided herein are lateral flow immunoassay devices and methods of using the same. Also provided herein are reader systems and devices configured to receive an immunoassay device of the disclosure and to provide an output based on the results of the immunoassay. Further, provided herein are kits comprising immunoassay devices and/or reader devices of the disclosure. The disclosed methods, devices, and kits may be useful for performing immunoassay tests on a sample, for example, to diagnose a disease or to provide information regarding a biological state or condition of a subject (e.g., fertility status). The disclosed methods, devices, and kits may be suitable for detecting the presence of a wide variety of analytes in a sample. In some cases, the disclosed methods, devices, and kits may be especially suited for detecting the presence of more than one analyte in a sample. In some cases, the disclosed methods, devices, and kits may detect the presence of more than one analyte in a sample with greater specificity and/or sensitivity than currently available immunoassay tests. In some cases, the devices, systems, methods, and kits provided herein may enable a user to perform a quantitative measurement with higher accuracy and wider dynamic range than currently available immunoassay devices.

As used in the specification and claims, the singular form “a”, “an” and “the” include plural references unless the context clearly dictates otherwise. For example, the term “a cell” includes a plurality of cells, including mixtures thereof.

As used herein, the term “about” a number refers to that number plus or minus 10% of that number. The term “about” a range refers to that range minus 10% of its lowest value and plus 10% of its greatest value.

### Immunoassay Test Devices

Provided herein are immunoassay test devices for the detection of one or more analytes in a sample. In one aspect, an immunoassay device is provided for determining the presence of a first analyte and a second analyte in a biological sample, the immunoassay device comprising: a test strip defining a flow path and comprising: a) at a first end, a sample zone configured to be contacted with a biological sample suspected of containing the first analyte and the second analyte; b) a labeling zone having absorbed thereon a mobilizable first detection reagent conjugated to a first fluorescent label and a mobilizable second detection reagent conjugated to a second fluorescent label, which the first detection reagent specifically binds to the first analyte thereby forming a first analyte-first detection reagent complex and the second detection reagent specifically binds to the second analyte thereby forming a second analyte-second detection reagent complex; c) a capture zone comprising a first capture region and a second capture region, wherein the first capture region has immobilized thereon a first capture reagent which specifically binds to the first detection reagent when the first detection reagent is not in a complex with the first analyte, and the second capture region has immobilized thereon a second capture reagent which specifically binds to the second analyte-second detection reagent complex, wherein a first optical signal from the first fluorescent label is capable of being detected at the first capture region and which the first optical signal decreases with increasing amounts of the first analyte present in the biological sample, and wherein a second optical signal from the second fluorescent label is capable of being detected at the second capture region and which the second optical signal increases with increasing amounts of the second analyte present in the biological sample.

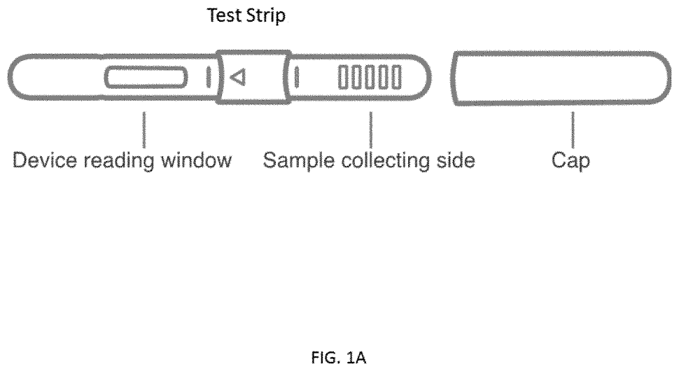

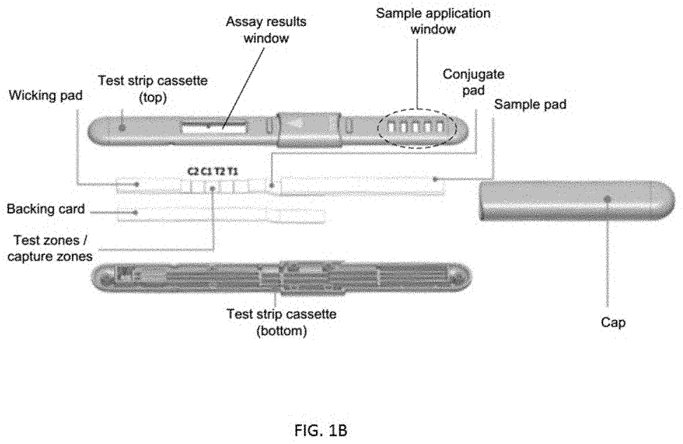

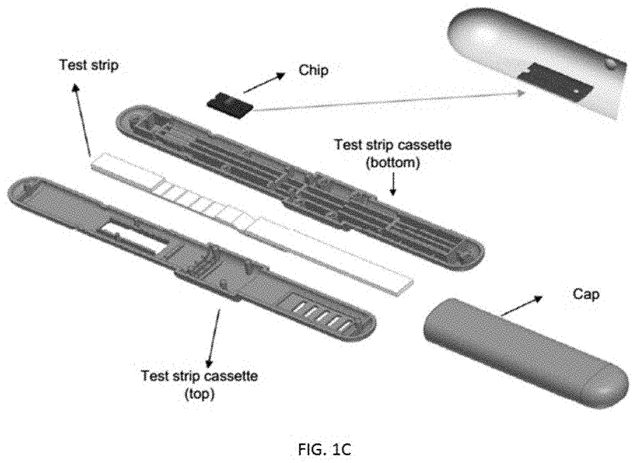

The test device may comprise a test strip for conducting the one or more immunoassays, as depicted in FIGS. 1A-C. The test strip may contain a plurality of reagents for conducting the one or more immunoassays. The reagents may be disposed on a surface of the test strip, for example, the reagents may be immobilized on a surface of the test strip, or may be adsorbed to the test strip. In some cases, reagents may be provided on the test strip in such a manner that they are mobilizable when contacted with a fluid. Generally, the test strip is composed of a material suitable for performing the one or more immunoassays, for providing an appropriate substrate for immobilizing or otherwise providing various reagents to perform the immunoassay, and for providing a means for lateral flow across the test strip. In some cases, the test strip is composed of a material comprising a plurality of capillary beds such that, when contacted with a fluid, the fluid is transported laterally across the test strip. Non-limiting examples of test strips may include porous paper, or a membrane polymer such as nitrocellulose, polyvinylidene fluoride, nylon, Fusion 5™, or polyethersulfone. The test strip may have arranged thereon a plurality of discrete regions, each having immobilized or otherwise provided thereon a variety of reagents for performing the immunoassay as described throughout the disclosure. The plurality of discrete regions may include, without limitation, a capture zone comprising one or more capture regions and/or a control zone comprising one or more control regions. The test strip may further define a flow path, with the plurality of discrete regions arranged laterally on the flow path, such that as the fluid flows along the test strip from a proximal end to a distal end, the immunoassay is conducted in a step-wise fashion.

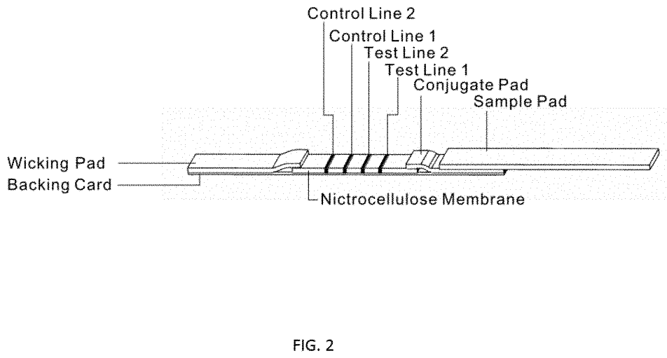

In various aspects, the test strip of the device may include a sample zone (also referred to herein as a sample pad) for receiving a fluid sample, as depicted in FIG. 1B. The sample zone may correspond to a portion or region of the test device that is configured to receive or accept a sample. Generally, the sample zone may be located at or near a first end of the test device. In some cases, the sample zone is located at a proximal end of the test device. The sample zone may be positioned on a proximal end of the flow path of the test strip such that a fluid sample may flow from the proximal end to the distal end of the test strip. A fluid sample may be applied to the sample zone by, e.g., inserting the proximal end of the device containing the sample zone into a container holding the fluid sample, by pipetting the fluid sample directly onto the sample zone, or by holding the proximal end of the device under a fluid stream (e.g., a urine stream), as depicted in FIG. 2. Generally, the sample is a fluid sample. In other cases, the sample is a solid sample that is modified to form a fluid sample, for example, dissolved or disrupted (e.g., lysed) in a liquid medium.

The sample zone may include a pad or other contact surface. In some cases, the pad may be composed of a woven mesh or a fibrous material such as a cellulose filter, polyesters, or glass fiber. The sample zone may further include, without limitation, pH and ionic strength modifiers such as buffer salts (e.g., Tris), viscosity enhancers to modulate flow properties, blocking and resolubilization agents (e.g., proteins (such as albumin), detergents, surfactants (such as Triton X-100, Tween-20), and/or filtering agents (e.g., for whole blood).

Generally, the sample applied to the sample zone is a fluid sample or a solid sample modified with a liquid medium. In various aspects, the sample is a biological sample. Non-limiting examples of biological samples suitable for use with the immunoassay devices of the disclosure include: whole blood, blood serum, blood plasma, urine, feces, saliva, vaginal secretions, semen, interstitial fluid, mucus, sebum, sweat, tears, crevicular fluid, aqueous humour, vitreous humour, bile, breast milk, cerebrospinal fluid, cerumen, enolymph, perilymph, gastric juice, peritoneal fluid, vomit, and the like. The biological sample can be obtained from a hospital, laboratory, clinical or medical laboratory. In some cases, the immunoassay test is performed by a clinician or laboratory technician. In other cases, the immunoassay test is performed by the subject, for example, at home.

The biological sample can be from a subject, e.g., a plant, fungi, eubacteria, archaebacteria, protist, or animal. The subject can be an organism, either a single-celled or multi-cellular organism. The subject can be cultured cells, which can be primary cells or cells from an established cell line, among others. Examples of cell lines include, but are not limited to, 293-T human kidney cells, A2870 human ovary cells, A431 human epithelium, B35 rat neuroblastoma cells, BHK-21 hamster kidney cells, BR293 human breast cells, CHO Chinese hamster ovary cells, CORL23 human lung cells, HeLa cells, or Jurkat cells. The sample can be isolated initially from a multi-cellular organism in any suitable form. The animal can be a fish, e.g., a zebrafish. The animal can be a mammal. The mammal can be, e.g., a dog, cat, horse, cow, mouse, rat, or pig. The mammal can be a primate, e.g., a human, chimpanzee, orangutan, or gorilla. The human can be a male or female. The sample can be from a human embryo or human fetus. The human can be an infant, child, teenager, adult, or elderly person. The female can be pregnant, suspected of being pregnant, or planning to become pregnant. The female can be ovulating. In some cases, the sample is a single or individual cell from a subject and the biological sample is derived from the single or individual cell. In some cases, the sample is an individual micro-organism, or a population of micro-organisms, or a mixture of micro-organisms and host cells.

In some cases, the biological sample comprises one or more bacterial cells. In some cases, the one or more bacterial cells are pathogens. In some cases, the one or more bacterial cells are infectious. Non-limiting examples of bacterial pathogens that can be detected include Mycobacteria (e.g. M. tuberculosis, M. bovis, M. avium, M. leprae, and M. africanum), Rickettsia, Mycoplasma, Chlamydia, and Legionella. Some examples of bacterial infections include, but are not limited to, infections caused by Gram positive bacillus (e.g., Listeria, Bacillus such as Bacillus anthracis, Erysipelothrix species), Gram negative bacillus (e.g., Bartonella, Brucella, Campylobacter, Enterobacter, Escherichia, Francisella, Hemophilus, Klebsiella, Morganella, Proteus, Providencia, Pseudomonas, Salmonella, Serratia, Shigella, Vibrio and Yersinia species), spirochete bacteria (e.g., Borrelia species including Borrelia burgdorferi that causes Lyme disease), anaerobic bacteria (e.g., Actinomyces and Clostridium species), Gram positive and negative coccal bacteria, Enterococcus species, Streptococcus species, Pneumococcus species, Staphylococcus species, and Neisseria species. Specific examples of infectious bacteria include, but are not limited to: Helicobacter pyloris, Legionella pneumophilia, Mycobacterium tuberculosis, Mycobacterium avium, Mycobacterium intracellulare, Mycobacterium kansaii, Mycobacterium gordonae, Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus viridans, Streptococcus faecalis, Streptococcus bovis, Streptococcus pneumoniae, Haemophilus influenzae, Bacillus antracis, Erysipelothrix rhusiopathiae, Clostridium tetani, Enterobacter aerogenes, Klebsiella pneumoniae, Pasteurella multocida, Fusobacterium nucleatum, Streptobacillus moniliformis, Treponema pallidium, Treponema pertenue, Leptospira, Rickettsia, and Actinomyces israelii, Acinetobacter, Bacillus, Bordetella, Borrelia, Brucella, Campylobacter, Chlamydia, Chlamydophila, Clostridium, Corynebacterium, Enterococcus, Haemophilus, Helicobacter, Mycobacterium, Mycoplasma, Stenotrophomonas, Treponema, Vibrio, Yersinia, Acinetobacter baumanii, Bordetella pertussis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Corynebacterium diphtheriae, Enterobacter sazakii, Enterobacter agglomerans, Enterobacter cloacae, Enterococcus faecalis, Enterococcus faecium, Escherichia coli, Francisella tularensis, Helicobacter pylori, Legionella pneumophila, Leptospira interrogans, Mycobacterium leprae, Mycobacterium tuberculosis, Mycobacterium ulcerans, Mycoplasma pneumoniae, Pseudomonas aeruginosa, Rickettsia rickettsii, Salmonella typhi, Salmonella typhimurium, Salmonella enterica, Shigella sonnei, Staphylococcus epidermidis, Staphylococcus saprophyticus, Stenotrophomonas maltophilia, Vibrio cholerae, Yersinia pestis, and the like.

The biological sample may comprise one or more viruses. Non-limiting examples of viruses include the herpes virus (e.g., human cytomegalomous virus (HCMV), herpes simplex virus 1 (HSV-1), herpes simplex virus 2 (HSV-2), varicella zoster virus (VZV), Epstein-Barr virus), influenza A virus and Hepatitis C virus (HCV) or a picornavirus such as Coxsackievirus B3 (CVB3). Other viruses may include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In some cases, the virus is an enveloped virus. Examples include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicella-zoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoonpox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta toro virus (PTV), Tacaribe virus and Tamiami virus. In some embodiments, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Birnavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses Al-22, 24 (CAl-22 and CA24, CA23 (echovirus 9)), human coxsackieviruses (Bl-6 (CBl-6)), human echoviruses 1-7, 9, 11-27, 29-33, vilyuish virus, simian enteroviruses 1-18 (SEV1-18), porcine enteroviruses 1-11 (PEVl-11), bovine enteroviruses 1-2 (BEV1-2), hepatitis A virus, rhinoviruses, hepatoviruses, cardio viruses, aphthoviruses and echoviruses. The virus may be phage. Examples of phages include, but are not limited to T4, T5, λ phage, T7 phage, G4, P1, φ6, Thermoproteus tenax virus 1, M13, MS2, Qβ, φX174, Φ29, PZA, Φ15, BS32, B103, M2Y (M2), Nf, GA-1, FWLBc1, FWLBc2, FWLLm3, B4. In some cases, the virus is selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus. In some cases, the virus is selected from a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus. In some cases, the virus is selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus. In some cases, the virus is selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O' nyong'nyong virus, and the chikungunya virus; a member of the Poxyiridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively).

The biological sample may comprise one or more fungi. Examples of infectious fungal agents include, without limitation Aspergillus, Blastomyces, Coccidioides, Cryptococcus, Histoplasma, Paracoccidioides, Sporothrix, and at least three genera of Zygomycetes. The above fungi, as well as many other fungi, can cause disease in pets and companion animals. The present teaching is inclusive of substrates that contact animals directly or indirectly. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans. Further examples of fungal infectious agent include, but are not limited to, Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

The biological sample may comprise one or more parasites. Non-limiting examples of parasites include Plasmodium, Leishmania, Babesia, Treponema, Borrelia, Trypanosoma, Toxoplasma gondii, Plasmodium falciparum, P. vivax, P. ovale, P. malariae, Trypanosoma spp., or Legionella spp. In some cases, the parasite is Trichomonas vaginalis.

In some cases, the biological sample is a sample taken from a subject infected with or suspected of being infected with an infectious agent (e.g., bacteria, virus). In some aspects, the biological sample comprises an infectious agent associated with a sexually-transmitted disease (STD) or a sexually-transmitted infection (STI). Non-limiting examples of STDs or STIs and associated infectious agents that may be detected with the devices and methods provided herein may include, Bacterial Vaginosis; Chlamydia (Chlamydia trachomatis); Genital herpes (herpes virus); Gonorrhea (Neisseria gonorrhoeae); Hepatitis B (Hepatitis B virus); Hepatitis C (Hepatitis C virus); Genital Warts, Anal Warts, Cervical Cancer (Human Papillomavirus); Lymphogranuloma venereum (Chlamydia trachomatis); Syphilis (Treponema pallidum); Trichomoniasis (Trichomonas vaginalis); Yeast infection (Candida); and Acquired Immunodeficiency Syndrome (Human Immunodeficiency Virus).

The sample can be from an environmental source or an industrial source. Examples of environmental sources include, but are not limited to, agricultural fields, lakes, rivers, water reservoirs, air vents, walls, roofs, soil samples, plants, and swimming pools. Examples of industrial sources include, but are not limited to clean rooms, hospitals, food processing areas, food production areas, food stuffs, medical laboratories, pharmacies, and pharmaceutical compounding centers. The sample can be a forensic sample (e.g., hair, blood, semen, saliva, etc.). The sample can comprise an agent used in a bioterrorist attack (e.g., influenza, anthrax, smallpox).

The biological sample can be from a subject (e.g., human subject) who is healthy. The biological sample can be from a pregnant female mammal. In some cases, the biological sample is taken from the pregnant female mammal at at least 4, 5, 6, 7, 8, 9, 10, 11, 12, 13, 14, 15, 16, 17, 18, 19, 20, 21, 22, 23, 24, 25, or 26 weeks of gestation. The biological sample may be taken from a female mammal during a menstrual cycle. In some cases, the biological sample is taken from a female mammal 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 days from the last menstrual period. The biological sample may be taken from a female mammal during an ovulation cycle. In some cases, the biological sample is taken from an ovulating female mammal 11, 12, 13, 14, 15, 16, 17, 18, 19, 20, or 21 days from the last menstrual period. The biological sample may be taken from a female mammal to determine a time of elevated fertility, for example, a period of time during the ovulation cycle in which the female mammal is most likely to become pregnant.

In some cases, more than one sample can be obtained from a subject or source and multiple immunoassay tests (e.g., utilizing multiple immunoassay devices) can be performed. In some cases, 2, 3, 4, 5, 6, 7, 8, 9, 10, 15, 20, 25, 30, 35, 40, 45, 50 or more samples can be obtained. In some cases, more than one sample may be obtained over a period of time, for example, to monitor disease progression or to monitor a biological state or condition (e.g., fertility status). Generally, the immunoassay devices of the disclosure are configured for one-time use (e.g., disposable).

In some cases, the subject is affected by a genetic disease, a carrier for a genetic disease or at risk for developing or passing down a genetic disease, where a genetic disease is any disease that can be linked to a genetic variation such as mutations, insertions, additions, deletions, translocation, point mutation, trinucleotide repeat disorders and/or single nucleotide polymorphisms (SNPs).

The biological sample can be from a subject who has a specific disease, disorder, or condition, or is suspected of having (or at risk of having) a specific disease, disorder or condition. For example, the biological sample can be from a cancer patient, a patient suspected of having cancer, or a patient at risk of having cancer. The cancer can be, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. The sample can be from the cancer and/or normal tissue from the cancer patient. In some cases, the sample is a biopsy of a tumor.

The biological sample can be processed to render it competent for performing any of the methods using any of the devices or kits provided herein. For example, a solid sample may be dissolved in a liquid medium or otherwise prepared as a liquid sample to facilitate flow along the test strip of the device. In such cases where biological cells or particles are used, the biological cells or particles may be lysed or otherwise disrupted such that the contents of the cells or particles are released into a liquid medium. Molecules contained in cell membranes and/or cell walls may also be released into the liquid medium in such cases. A liquid medium may include water, saline, cell-culture medium, or any solution and may contain any number of salts, surfactants, buffers, reducing agents, denaturants, preservatives, and the like.

Generally, the sample contains or is suspected of containing one or more analytes. In various aspects, the sample may contain at least a first analyte and a second analyte. The term “analyte” as used herein may refer to any substance that is to be analyzed using the methods and devices provided herein. The immunoassay device may be configured to detect the presence of 1, 2, 3, 4, 5, 6, 7, 8, 9, 10 or more analytes in a sample.

Non-limiting examples of analytes may include proteins, haptens, immunoglobulins, hormones, polynucleotides, steroids, drugs, infectious disease agents (e.g., of bacterial or viral origin), drugs of abuse, environmental agents, biological markers, and the like. In one case, the immunoassay detects at least a first analyte, wherein the first analyte is luteinizing hormone (LH). In another case, the immunoassay detects at least a first analyte, wherein the first analyte is human chorionic gonadotropin (hCG). In another case, the immunoassay detects at least a first analyte and a second analyte, wherein the first analyte is estrone-3-glucoronide (E3G) and the second analyte is luteinizing hormone (LH). In another case, the immunoassay detects at least a first analyte and a second analyte, wherein the first analyte is a surface antigen on a first viral particle (e.g., Influenza A) and the second analyte is a surface antigen on a second viral particle (e.g., Influenza B). In another case, the immunoassay detects at least a first analyte, wherein the first analyte is 25-hydroxyvitamin D, 25-hydroxyvitamin D2 [25(OH)D₂], or 25-hydroxyvitamin D3 [25(OH)D₃]. In another case, the immunoassay detects at least a first analyte and a second analyte, wherein the first analyte is triiodothyronine (T3) and the second analyte is thyroxine (T4). In another case, the immunoassay detects at least a first analyte, wherein the first analyte is an allergen. Non-limiting examples of allergens may include: Balsam of Peru, fruit, rice, garlic, oats, meat, milk, peanuts, fish, shellfish, soy, tree nuts, wheat, hot peppers, gluten, eggs, tartrazine, sulfites, tetracycline, phenytoin, carbamazepine, penicillin, cephalosporins, sulfonamides, non-steroidal anti-inflammatories (e.g., cromolyn sodium, nedocromil sodium, etc.), intravenous contrast dye, local anesthetics, pollen, cat allergens, dog allergens, insect stings, mold, perfume, cosmetics, semen, latex, water, house dust mites, nickel, gold, chromium, cobalt chloride, formaldehyde, photographic developers, fungicide, dimethylaminopropylamine, paraphenylenediamine, glyceryl monothioglycolate, toluenesulfonomide formaldehyde.

The device may be used to test for the presence or absence of at least a first analyte and a second analyte in a sample. In some cases, the device may be used to determine an amount or a relative amount of at least a first and second analyte in a sample.

The presence or absence of analytes may be indicative of a disease or disorder in a subject. The presence or absence of analytes may be indicative of a biological state or condition of a subject. In one example, the presence or absence of analytes may indicate the fertility of a female mammal. The presence or absence of analytes may indicate that a female mammal has an elevated fertility or is at a particular period of time in an ovulation cycle. In some cases, the presence or absence of analytes indicates that a female mammal is pregnant. In some cases, the presence or absence of analytes indicates the gestation time of a female mammal. In some cases, the presence or absence of analytes indicates that a subject has or is at risk of developing a disease. In some cases, the presence or absence of analytes indicates that a subject has a disorder (e.g., thyroid disorder). In some cases, the presence or absence of analytes indicates that a subject has a deficiency (e.g., vitamin deficiency). In some cases, the presence or absence of analytes indicates that a product (e.g., a food or drink product) contains an allergen.

The test strip may further comprise a labeling zone, also referred to herein as a conjugate pad, as depicted in FIG. 1B. The labeling zone may be composed of, for example, glass fibers, cellulose filters, or surface-modified polyester. The labeling zone may be positioned on a flow path of the test strip. In a particular case, the labeling zone is positioned on a flow path of the test strip downstream from the sample zone such that the sample flows from the sample zone to the labeling zone. The labeling zone may comprise at least a first detection reagent and a second detection reagent. The first detection reagent and the second detection reagent may be adsorbed on a surface of the labeling zone. The first detection reagent and the second detection reagent may be freely mobile or mobilizable when in a wet state, for example, when a liquid saturates the labeling zone. The first detection reagent may specifically bind to a first analyte, when present in the sample, thereby forming a first analyte-first detection reagent complex. The second detection reagent may specifically bind to a second analyte, when present in the sample, thereby forming a second analyte-second detection reagent complex. Generally, the first detection reagent and the second detection reagent have specific binding activity for the first and second analytes, respectively. For example, the first detection reagent may specifically or selectively bind to the first analyte, but not the second analyte, and likewise, the second detection reagent may specifically or selectively bind to the second analyte, but not the first analyte. In situations where it is desirable to detect the presence of more than two analytes, additional detection reagents may be utilized with specific binding activity for the additional analytes. Essentially any number of detection reagents may be used to detect a desirable number of analytes, but generally, the device will include at least a first detection reagent and a second detection for the detection of a first analyte and a second analyte, respectively.

The detection reagents may include an antibody or antibody fragment, an antigen, an aptamer, a peptide, a small molecule, a ligand, a molecular complex or any combination thereof. Essentially, the first and second detection reagents may be any reagents that have specific binding activity for the first analyte and the second analyte, respectively. In particular cases, the first detection reagent and the second detection reagent are antibodies or antibody fragments that specifically bind to epitopes present on the first analyte and second analyte, respectively. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. In some cases, the antibody is an antigen-binding antibody fragment such as, for example, a Fab, a F(ab′), a F(ab′)2, a Fd chain, a single-chain Fv (scFv), a single-chain antibody, a disulfide-linked Fv (sdFv), a fragment comprising either a VL or VH domain, or fragments produced by a Fab expression library. Antigen-binding antibody fragments, including single-chain antibodies, can comprise the variable region(s) alone or in combination with the entirety or a portion of the following: hinge region, CH1, CH2, CH3 and CL domains. Also, antigen-binding fragments can comprise any combination of variable region(s) with a hinge region, CH1, CH2, CH3 and CL domains. Antibodies and antibody fragments may be derived from a human, rodent (e.g., mouse and rat), donkey, sheep, rabbit, goat, guinea pig, camelid, horse, or chicken. Various antibodies and antibody fragments may be designed to selectively bind essentially any desired analyte. Methods of generating antibodies and antibody fragments are well known in the art.

The terms “selective” or “specific” binding may be used herein interchangeably. Generally speaking, a ligand that selectively or specifically binds to a target means that the ligand has a high binding affinity for its target, and a low binding affinity for non-target molecules. The dissociation constant (K_{d}) may be used herein to describe the binding affinity of a ligand for a target molecule (e.g., an analyte). The dissociation constant may be defined as the molar concentration at which half of the binding sites of a target molecule are occupied by the ligand. Therefore, the smaller the K_{d}, the tighter the binding of the ligand to the target molecule. In some cases, a ligand has a dissociation constant (K_{d}) for a target molecule of less than 1 mM, less than 100 μM, less than 10 μM, less than 1 μM, less than 100 nM, less than 50 nM, less than 25 nM, less than 10 nM, less than 5 nM, less than 1 nM, less than 500 pM, less than 100 pM, less than 50 pM, or less than 5 pM.

In various aspects, detection reagents may be conjugated or otherwise attached to a detectable label. For example, the first detection reagent may be conjugated to a first detectable label and the second detection reagent may be conjugated to a second detectable label. The detectable label may be a fluorophore, an enzyme, a quencher, an enzyme inhibitor, a radioactive label, one member of a binding pair or any combination thereof. In some cases, the first and/or second detectable labels are fluorescent molecules, e.g., fluorophores. Non-limiting examples of fluorophores suitable for use with the immunoassay devices may include: fluorescein (FITC) and fluorescein derivatives such as FAM, VIC, and JOE, 5-(2′-aminoethyl)aminonaphthalene-1-sulphonic acid (EDANS), coumarin and coumarin derivatives, Lucifer yellow, NED, Texas red, tetramethylrhodamine, tetrachloro-6-carboxyfluoroscein, 5 carboxyrhodamine, cyanine dye, Alexa Fluor 350, Alexa Fluor 647, Oregon Green, Alexa Fluor 405, Alexa Fluor 680, Alexa Fluor 488, Alexa Fluor 750, Cy3, Alexa Fluor 532, Pacific Blue, Pacific Orange, Alexa Fluor 546, Tetramethylrhodamine (TRITC), Alexa Fluor 555, BODIPY FL, Texas Red, Alexa Fluor 568, Pacific Green, Cy5, Alexa Fluor 594, Super Bright 436, Super Bright 600, Super Bright 645, Super Bright 702, DAPI, SYTOX Green, SYTO 9, TO-PRO-3, Propidium Iodide, Qdot 525, Qdot 565, Qdot 605, Qdot 655, Qdot 705, Qdot 800, R-Phycoerythrin (R-PE), Allophycocyanin (APC), cyan fluorescent protein (CFP) and derivatives thereof, green fluorescent protein (GFP) and derivatives thereof, red fluorescent protein (RFP) and derivatives thereof, and the like. Any fluorophore with an excitation wavelength of between about 300 nm and about 900 nm is envisioned herein.

In some cases, the first and second detectable labels are the same detectable label. In other cases, the first and second detectable labels are different detectable labels. The first detection reagent and the second detection reagent may be present at the labeling zone in an amount that is in excess of the amount of first analyte and second analyte, respectively, present in the original sample. For example, the first and/or second detection reagents may be present at the labeling zone at an amount at least 2×, 3×, 4×, 5×, 6×, 7×, 8×, 9×, 10×, 15×, 20×, 25×, 30×, 35×, 40×, 45×, 50×, 55×, 60×, 65×, 70×, 75×, 80×, 85×, 90×, 95×, 100×, 150×, 200×, 250×, 300×, 350×, 400×, 450×, 500×, 550×, 600×, 650×, 700×, 750×, 800×, 850×, 900×, 950×, 1000×, or greater than the amount of first and/or second analyte present in the sample.

The labeling zone may further comprise additional chemicals, including, but not limited to, bovine serum albumin (BSA), polyvinylpyrrolidine 40 (PVP40), Triton X-100, sucrose, Tween-20, Tris, fetal bovine serum (FBS), or any combination thereof.

The test strip may further comprise a capture zone, also referred to herein as a test zone, as depicted in FIG. 1B. The capture zone may be positioned on the flow path of the test strip. In some cases, the capture zone is positioned on the flow path of the test strip downstream of the labeling zone, such that the sample can flow from the labeling zone to the capture zone. The capture zone may comprise one or more discrete capture regions. Generally, the number of capture regions employed will depend on the number of analytes to be detected. For example, if two analytes are to be detected, the device will have two capture regions, if three analytes are to be detected, the device will have three capture regions, and so on.

In various aspects, the capture zone comprises at least a first capture region and a second capture region. The first capture region may have immobilized thereon a first capture reagent. Methods of immobilizing reagents to a test strip are widely known in the art and essentially any method may be used. In some examples, the first capture reagent may be immobilized to the first capture region by direct adsorption to the membrane or by covalently attaching the reagent to a linker molecule. The first capture region may be configured for the detection of the first analyte present in the sample. In a particular aspect, the first capture region may be configured to perform a competitive binding-like assay as described herein. In a particular aspect, the first capture reagent may specifically bind to non-complexed first detection reagent (i.e., when the first detection reagent has not formed a complex with the first analyte at the labeling zone). In such cases, the first capture reagent does not bind to the first detection reagent when the first detection reagent is bound to the first analyte (i.e., the first capture reagent does not bind to the first analyte-first detection reagent complex, when formed). In further instances, the first capture reagent may not displace the first analyte from the first detection reagent when the first analyte is bound to the first detection reagent. Thus, the amount of first analyte present in the original sample may determine the amount of first detection reagent that is capable of binding to the first capture reagent at the first capture region. For example, when the amount of first analyte present in the original sample is low or the first analyte is absent from the sample, the first detection reagent may be mostly in a non-complexed state when it reaches the capture region, and therefore more non-complexed first detection reagent may be available for binding to the first capture region. When the amount of first analyte present in the original sample is high, the first detection reagent may be mostly present at the first capture region in a complex with the first analyte, and therefore, less first detection reagent may be available for binding to the first capture reagent.

In various aspects, the first capture region is configured such that a first optical signal (e.g., a fluorescent signal) is capable of being detected at the first capture region. For example, the device may be insertable into a reader device as described herein for detection of a first optical signal at the first capture region. The first optical signal may be a readout for the amount of first analyte present in the sample, for example, by detecting the amount of first detection reagent bound to the first capture reagent as described above. In such cases, the first optical signal increases when the amount of first analyte present in the sample is low, and the first optical signal decreases when the amount of first analyte present in the sample is high. The optical signal at the first capture region may be proportional to the amount of first analyte present in the sample (e.g., directly proportional, inversely proportional, exponentially proportional, logarithmically proportional, etc.).

In various aspects, the capture zone further comprises a second capture region. The second capture region may have immobilized thereon a second capture reagent. Methods of immobilizing reagents to a test strip are widely known in the art and essentially any method may be used. In some examples, the second capture reagent may be immobilized to the second capture region by direct adsorption to the membrane or by covalently attaching the reagent to a linker molecule. The second capture region may be configured for the detection of the second analyte present in the sample. In a particular aspect, the first capture region may be configured to perform a sandwich binding assay as described herein. In a particular aspect, the second capture reagent may specifically bind to the second analyte-second detection reagent complex. In such cases, the second capture reagent does not bind to the second analyte or the second detection reagent, unless they are present in a complex with one another. Thus, the amount of second analyte present in the original sample will determine the amount of second analyte-second detection reagent complex that is capable of binding to the second capture reagent at the second capture region. For example, when the amount of second analyte present in the original sample is low or is absent in the sample, the second detection reagent may be mostly in a non-complexed state when it reaches the second capture region, less second analyte-second detection reagent complex may be formed at the labeling zone, and less second analyte-second detection complex may be available for binding to the second capture reagent at the second capture region. When the amount of second analyte present in the original sample is high, the second detection reagent may be mostly present at the second capture region in a complex with the second analyte, and therefore, more second analyte-second detection reagent complex may be available for binding to the second capture reagent at the second capture region.

In various aspects, the second capture region is configured such that a second optical signal (e.g., a fluorescent signal) is capable of being detected at the second capture region. For example, the device may be insertable into a reader device as described herein for detection of a second optical signal at the second capture region. The second optical signal may be a read-out for the amount of second analyte present in the sample, for example, by detecting the amount of second analyte-second detection reagent complex bound to the second capture reagent as described above. In such cases, the second optical signal increases when the amount of second analyte present in the sample is high, and the second optical signal decreases when the amount of second analyte present in the sample is low. The optical signal at the second capture region may be proportional to the amount of second analyte present in the sample (e.g., directly proportional, inversely proportional, exponentially proportional, logarithmically proportional, etc.).

Generally, the capture zone comprises at least a first capture region and a second capture region. In some instances, the first capture region and second capture region are present on a flow path of the test strip. In one example, the first capture region is upstream of the second capture region. In another example, the first capture region is downstream of the second capture region. When the device utilizes two capture regions, it should be understood that the order of the capture regions is unimportant, so long as one capture region utilizes a sandwich binding assay and the other utilizes a competitive binding assay as described above. In some cases, the capture zone may comprise more than two capture regions, for example, the capture zone may comprise three, four, five, six, seven, eight, nine, ten or more than ten capture regions. Each capture region should be capable of detecting the presence of a different analyte, thus, if the device is configured to detect three analytes, three capture regions should be utilized, and so on.

In various aspects, the test device may further comprise a control zone, as depicted in FIG. 1B. The control zone may comprise at least a first control region and a second control region. The first and second control regions may be positioned on a flow path of the test strip. Generally, the first and second control regions are positioned on a flow path of the test strip downstream of the capture zone such that the sample may flow (e.g., by capillarity) from the capture zone to the control zone. The first and second control regions may have, immobilized thereon, a control reagent. Methods of immobilizing reagents to a test strip are widely known in the art and essentially any method may be used. In some examples, the control reagent may be immobilized to the first and second control regions by direct adsorption to the membrane or by covalently attaching the reagent to a linker molecule. In some instances, the control reagent binds to both the first and the second detection reagents. It should be understood that the control reagent is to be selected based on the identity of the first and second detection reagents and that essentially any ligand with binding affinity for the first and second detection reagents may be utilized. In one particular aspect, the first and second detection reagents are mouse IgGs and the control reagent is an anti-mouse IgG antibody. The first and second control regions may be configured to capture excess first and second detection reagents that did not bind to the capture zone.

In one example, when the amount of first analyte and second analyte are high in the sample, the amount of first detection reagent present at the first control region and available for binding the control reagent may be high (i.e., because the amount of first detection reagent bound to the first capture region is low), whereas the amount of second detection reagent present at the first control region and available for binding the control reagent may be low (i.e., because the amount of second detection reagent bound to the second capture region is high). In such situations, the signal generated at the first control region will mostly constitute signal generated from the first detection reagent (with a smaller amount being generated by the second detection reagent). In contrast, when the amount of first analyte and second analyte are low or absent from the sample, the amount of first detection reagent present at the first control region and available for binding the control reagent may be low (i.e., because the amount of first detection reagent bound to the first capture region is high), whereas the amount of second detection reagent present at the first control region and available for binding the control reagent may be high (i.e., because the amount of second detection reagent bound to the second capture region is low). In such situations, the signal generated at the first control region will mostly constitute signal generated from the second detection reagent (with a smaller amount being generated by the second detection reagent).

In various aspects, the test device may further comprise a second control region positioned on a flow path of the test strip downstream from the first control region such that the sample flows (e.g., by capillarity) from the first control region to the second control region. The second control region may have, immobilized thereon, a second capture reagent that binds to both the first and second detection reagents. In some cases, the second capture reagent is the same as the first capture reagent. In a particular example, the second capture reagent is anti-mouse IgG antibody. The second control region may be configured to capture any excess first and/or second detection reagent present in the control zone. It should be understood that when the sample is flowed from the first control region to the second control region, the first and second detection reagents will be depleted from the sample when it is passed through the first control region, and therefore, in some embodiments, the signal generated at the second control region may be smaller than the signal generated at the first control region.

In some cases, the first control reagent may be provided at an amount greater than the second control reagent. For example, the amount of first control reagent may be present at the first control region at an amount 1.5×, 2.0×, 2.5×, 3.0×, 3.5×, 4.0×, 4.5×, 5.0×, 5.5×, 6.0×, 6.5×, 7.0×, 7.5×, 8.0×, 8.5×, 9.0×, 9.5×, 10.0× or greater than the amount of second control reagent present at the second control region. In other cases, the amount of first control reagent is equal to the amount of second control reagent. In yet other cases, the amount of second control reagent is greater than the amount of first control reagent. For example, the amount of second control reagent may be present at the second control region at an amount 1.5×, 2.0×, 2.5×, 3.0×, 3.5×, 4.0×, 4.5×, 5.0×, 5.5×, 6.0×, 6.5×, 7.0×, 7.5×, 8.0×, 8.5×, 9.0×, 9.5×, 10.0× or greater than the amount of first control reagent present at the first control region. The amounts of first control reagent and second control reagent may be customized or tailored to increase the sensitivity or precision of the immunoassay.

The immunoassay devices of the disclosure may further include one or more additional features, as depicted in FIGS. 1B and 1C. The immunoassay device may further include a wicking pad, generally located at the distal end of the immunoassay device, downstream from the test strip. The wicking pad may be composed of, e.g., filter paper. Other features of the immunoassay device may include a test strip cassette for supporting and/or protecting the test strip. The cassette may be composed of a sturdy material such as plastic (e.g., high-impact polystyrene). The cassette may, e.g., prevent a user from applying the sample anywhere other than the sample pad, may protect the device from inadvertent splashing of a sample onto the test strip (e.g., when the device is applied to a urine stream), and to protect the sensitive areas of the test strip (e.g., the capture and control zones). The cassette may include various openings or windows along the device. For example, the cassette may include, at a proximal end, a sample application window for applying a fluid sample to the sample pad. The cassette may further include an assay results window for visualization of the assay results. The assay results window may be positioned on the device directly above the capture zone and control zone such that a detectable signal can be visualized or read (e.g., by a diagnostic test system). The cassette may be of a certain size and shape so as to be compatible with a diagnostic test device of the disclosure, such that the cassette may be inserted into a port or receiver of a diagnostic test device. The immunoassay device may further include a cap composed of, e.g., polypropylene, designed to be inserted over the proximal end of the device to cover the sample application window. With reference to FIG. 1C, the immunoassay device may further include a readable chip. The readable chip may be positioned on the underside of the device, for example, embedded within or otherwise attached to the bottom of the test strip cassette. The readable chip may be configured to be in electronic communication with a diagnostic test system, after the device is inserted into the diagnostic test system. The readable chip may be recognized by the diagnostic test system such that upon insertion of the immunoassay device into the diagnostic test system, the diagnostic test system automatically performs one or more defined operations.

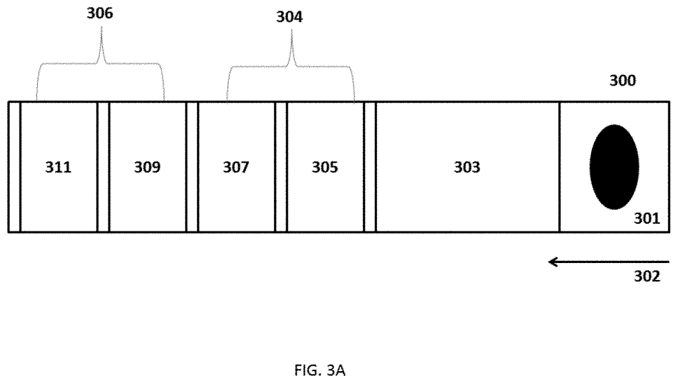

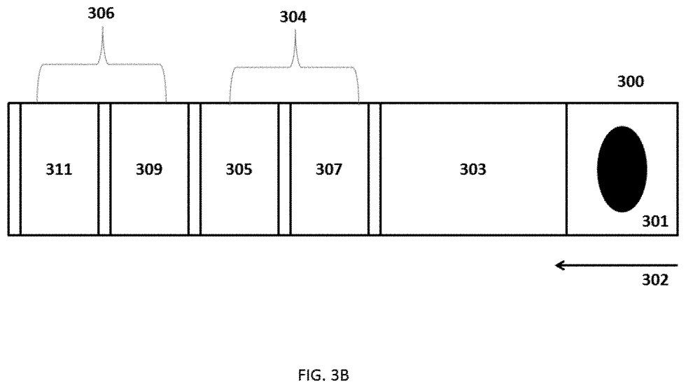

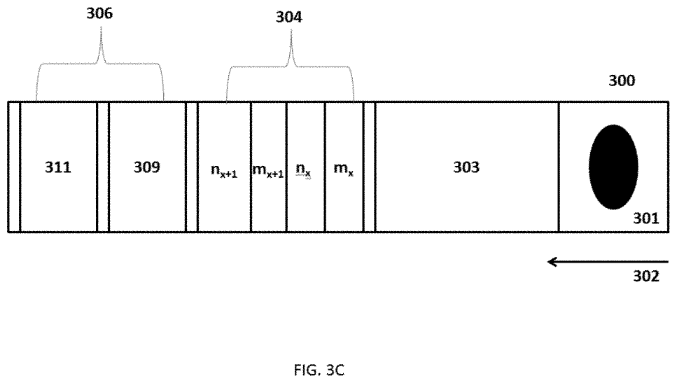

FIGS. 3A & B depict non-limiting examples of a test device comprising a test strip 300 in accordance with various embodiments as described herein. The test device may comprise various zones and/or regions for conducting an immunoassay. The test strip of the device may define a flow path. The various zones and/or regions for conducting immunoassays in accordance with the disclosure may be positioned along a flow path of the test strip such that a fluid sample may be flowed (e.g., by capillarity) from a first end of the test strip to a second end of the test strip, as depicted in FIGS. 3A & B by the directional arrow 302. The test device may include a sample zone 301. The sample zone may be positioned at a first end of the test strip and may be configured to receive a sample as described herein, such as by pipetting a sample onto the sample zone or by contacting the sample zone with a sample contained in a container. The test device may further comprise a labeling zone 303 (e.g., a conjugate pad) positioned downstream of the sample zone on the flow path of the test strip. The labeling zone 303 may contain one or more detection reagents for labeling one or more analytes when present in the sample. The test device may further comprise a capture zone 304 downstream on the flow path of the test strip from the capture zone 301. The capture zone may further comprise one or more capture regions, for example, a first capture region 305 and a second capture region 307. As described herein, each of the one or more capture regions may contain a capture reagent for performing an immunoassay. The immunoassay may be a sandwich-based immunoassay or a competitive binding-based immunoassay. In some cases, the first capture region 305 contains reagents suitable to perform a competitive binding-based immunoassay and the second capture region 307 contains reagents suitable to perform a sandwich-based immunoassay. In other cases, the first capture region 305 contains reagents suitable to perform a sandwich-based immunoassay and the second capture region 307 contains reagents suitable to perform a competitive binding-based immunoassay. As depicted in FIG. 3A, the first capture region 305 may be positioned upstream of the second capture region 307 on the flow path of the test strip. As depicted in FIG. 3B, the first capture region 305 may be positioned downstream of the second capture region 307 on the flow path of the test strip. The test device may further comprise a control zone 306 positioned downstream from the capture zone 304 on the flow path of the test strip. The control zone may further comprise one or more control regions, for example, a first control region 309 and a second control region 311. The second control region 311 may be positioned downstream of the first control region 309 on the flow path of the test strip. With reference to FIG. 3C, the immunoassay device may contain a plurality of first capture regions (m) and a plurality of second capture regions (n).

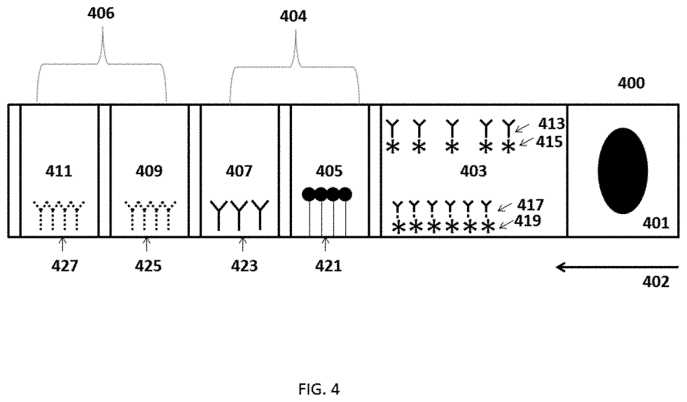

FIG. 4 depicts a non-limiting example of immunoassay reagents provided on a test device comprising a test strip 400 in accordance with the disclosure. The test device may comprise a sample zone 401 positioned at a first end of the device as described herein. The test device may further comprise a labeling zone 403 positioned downstream of the sample zone 401 on a flow path 402 of the test strip. The labeling zone 403 may comprise a mobilizable first detection reagent 413 conjugated to a first detectable label 415. The first detection reagent may be capable of specifically binding to a first analyte when present in the sample. The labeling zone 403 may further comprise a mobilizable second detection reagent 417 conjugated to a second detectable label 419. The second detection reagent may be capable of specifically binding to a second analyte when present in the sample. The test device may further comprise a capture zone 404 comprising a first capture region 405 and a second capture region 407. The first capture region 405 may have, immobilized thereon, a first capture reagent 421. The first capture reagent may be, e.g., an antigen. The second capture region 407 may have, immobilized thereon, a second capture reagent 423. The second capture reagent may be, e.g., an antibody or antibody fragment. The test device may further comprise a control zone 406 comprising a first control region 409 and a second control region 411. The first control region 409 may have, immobilized thereon, a first control reagent 425. The first control reagent may be, e.g., an antibody or antibody fragment. The second control region 411 may have, immobilized thereon, a second control reagent 427. The second control reagent may be, e.g., an antibody or antibody fragment.

### Reader Devices and Systems

Further provided herein are reader devices and systems suitable for use with the immunoassay devices described herein.

In one aspect, a diagnostic test system is provided comprising: a housing, comprising: a) a port for receiving an assay device, said assay device comprising two or more capture regions; b) a reader comprising: i) one or more light sources for illuminating said two or more capture regions; ii) one or more light detectors for detecting optical signals from said two or more capture regions; and c) a data analyzer having one or more processors configured to: A) receive said optical signals; and B) determine an amount of at least a first analyte and a second analyte present in a biological sample based on said optical signals, wherein an optical signal of a first of said two or more capture regions increases with decreasing amounts of said first analyte present in said biological sample, and an optical signal of a second of said two or more capture regions increases with increasing amounts of said second analyte present in said biological sample.

The diagnostic test system may include a housing for containing the components of the system. The housing can be constructed of any suitable material. The housing may be configured to receive an immunoassay device of the disclosure. For example, the housing may include a port or opening for receiving the immunoassay device. The system may further include, contained within the housing, a reader device. The reader device may include one or more light sources for illuminating the immunoassay device or a region of the immunoassay device. In one non-limiting example, the one or more light sources are configured to illuminate the capture zone of an immunoassay device of the disclosure. The type of light source suitable for use with the immunoassay devices will depend on the chemistry of the immunoassay device. In one particular example, the one or more light sources are used to illuminate a detectable label provided by the immunoassay device. In a particular example, the detectable label provided on the immunoassay device is a fluorophore, and therefore, the one or more light sources of the reader device should include a fluorescent light source (e.g., a light-emitting diode (LED)). It is to be understood that the wavelength of light provided by the light source of the reader device should be selected based on the excitation wavelength of the detectable label, and can readily be selected by a person of skill in the art.

The reader may be configured to illuminate the capture zone and/or the control zone of an immunoassay device of the disclosure. For example, the reader may be configured to illuminate the first capture region, the second capture region, the first control region, the second control region, or any combination thereof. In some cases, the reader is configured to scan across the test strip of an immunoassay device. In such cases where the immunoassay device utilizes a single fluorophore, the reader may contain a single fluorescent light source. In cases where the immunoassay device utilizes more than one fluorophore, the reader may contain more than one fluorescent light source.

The reader may further comprise one or more light detectors (e.g., a photodetector) for detecting optical signals from the immunoassay device. Generally speaking, the one or more light detectors should be capable of distinguishing between emitted light at a first discrete position and a second discrete position on the immunoassay device. This may be accomplished by, e.g., the one or more light sources scanning across the test strip of the immunoassay device and determining the position of the emitted light on the immunoassay device.

The diagnostic test device may further comprise a data analyzer. The data analyzer may have one or more processors configured to receive an optical signal. In some cases, the data analyzer is in operable communication with a reader device. The data analyzer may be configured to determine an amount of analytes present in a sample, for example, by measuring an amount of optical signal produced at the capture zone of an immunoassay device. For example, the data analyzer may be configured to calculate the area under the curve of a signal intensity plot. The data analyzer may further be configured to determine the differences between signal intensities among the multiple discrete regions on the test strip. For example, the data analyzer may be configured to determine the difference between the signal intensity at the first capture region and the signal intensity at the second control region. The data analyzer may further be configured to determine the difference between the signal intensity at the second capture region and the signal intensity at the first control region. The data analyzer may further be configured to calculate an amount or concentration of the analytes present in the sample. The data analyzer may be further configured to detect a binary optical pattern.

The binary optical pattern can be generated by two fluorescent materials which excitation and/or emission spectrum differs in wavelength. In some cases, the binary optical pattern can be generated by one fluorescent material and one light absorbent material. The detection reagents may be conjugated with the two types of materials respectively and can be captured in the same capture zone, such that the capture zone may generate two different optical signal patterns in the data analyzer.

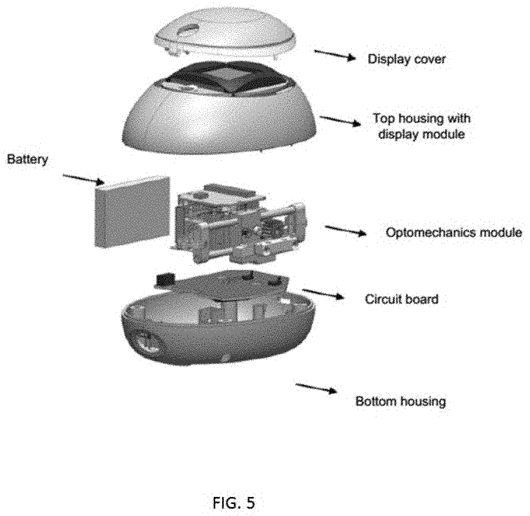

FIG. 5 depicts an exploded view of an exemplary diagnostic test system of the disclosure. The system may comprise a housing for containing the electronic components of the system. The system may have a top housing and a bottom housing. The top housing may comprise a display module for displaying the results of an immunoassay as described herein. The system may further comprise a display cover. The system may further comprise a battery. The system may further comprise an optomechanics module. The optomechanics module may comprise the one or more light sources and one or more light detectors as described above. The system may further comprise a circuit board containing electronic components.

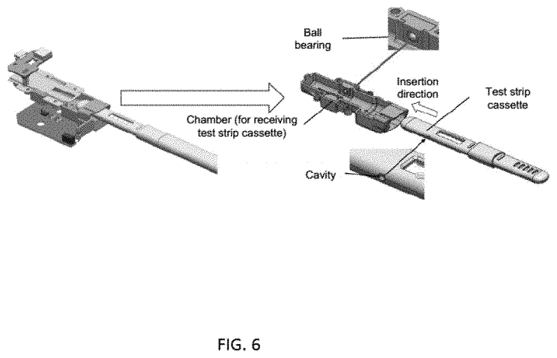

FIG. 6 depicts a non-limiting example of a receiving port or chamber of a diagnostic test system for receiving an immunoassay test device of the disclosure. The cassette or housing of the immunoassay test device may include a cavity. The chamber or receiving port of the diagnostic test system may include a ball bearing contained within the inner wall of the chamber. The ball bearing may hook or latch into the cavity of the test device, thereby locking the immunoassay test device into the receiving chamber of the diagnostic test system.

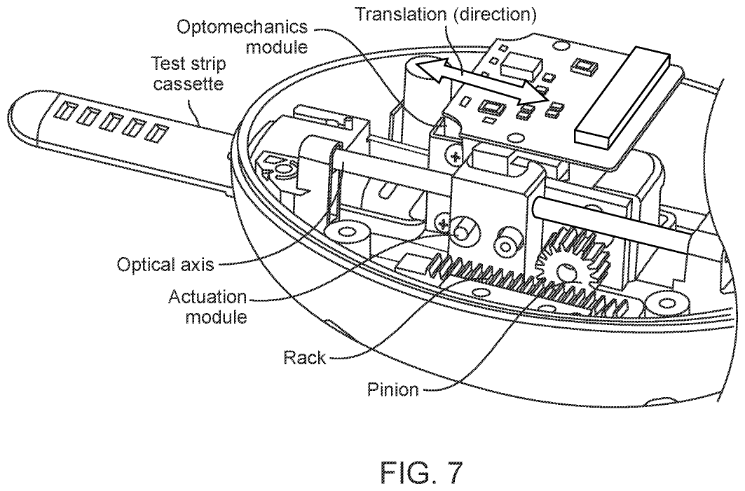

FIG. 7 depicts inner components of a diagnostic test system, in accordance with embodiments of the disclosure. The diagnostic test system may include an optomechanics module comprising the one or more light sources for illuminating the test strip of the immunoassay device. The optomechanics module may be movable across an optical axis such that the optomechanics module moves laterally across the test strip of the immunoassay device, thereby scanning the test strip. The diagnostic test system may further comprise an actuation module. The actuation module may comprise one or more motors configured to actuate/move the optomechanics module. In some embodiments, the motors may be coupled to a rack and pinion mechanism that is configured to translate the optomechanics module along one or more directions. For example, the optomechanics module can be translated along a longitudinal axis of the test strip of the immunoassay device. The direction(s) of translation may or may not be orthogonal to an optical axis of the optomechanics module. The direction(s) of translation may be parallel to the longitudinal axis of the test strip, and the optical axis may be orthogonal to the longitudinal axis or a planar surface of the test strip. In some cases, the direction(s) of translation need not be parallel to the longitudinal axis of the test strip, and the optical axis need not be orthogonal to the longitudinal axis (or a planar surface) of the test strip. For example, the direction(s) of translation and/or the optical axis may be at an oblique angle relative to the longitudinal axis of the test strip.

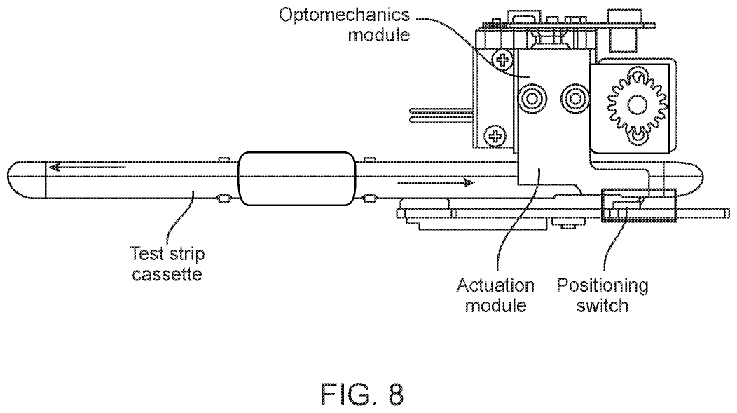

FIG. 8 depicts an immunoassay device inserted into the receiving chamber of a diagnostic test system of the disclosure. The diagnostic test system may include a positioning switch. The positioning switch may be configured to switch between an open position and a closed position. The positioning switch may be in the open position when the immunoassay device is not inserted into the receiving chamber. The position switch may be in the closed position when the immunoassay device is inserted into the receiving chamber and located at a predetermined position within the receiving chamber. The immunoassay device may be located at the predetermined position, for example when a cavity on the test strip cassette engages a ball-bearing within the chamber (see FIG. 6). The positioning switch may be configured to activate the actuation module and the optomechanics module to scan the test strip on the immunoassay device when the positioning switch is in the closed position. In some cases, the scan of the test strip may be terminated if the scan has been completed, or when the positioning switch has been moved to the open position.

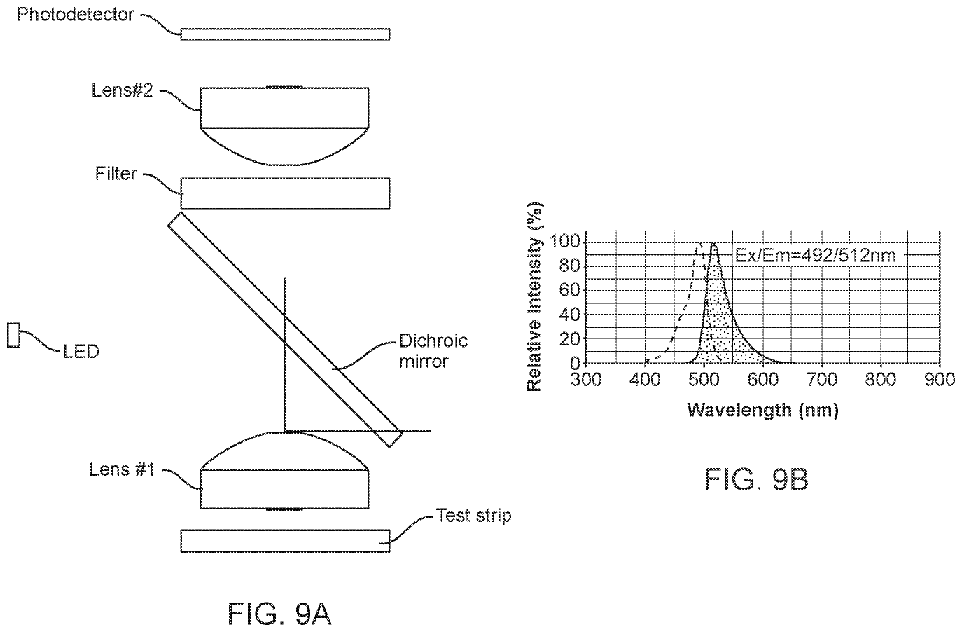

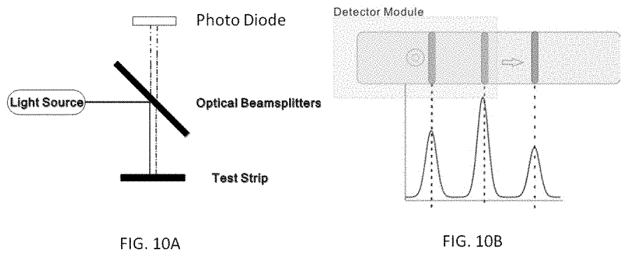

FIG. 9A depicts a non-limiting example of an optical configuration suitable for use with the diagnostic test system and positioning of the optics above a test strip of an immunoassay device. The optical configuration may include a light source (e.g., a light-emitting diode (LED) for illuminating the test strip. The optical configuration may further include one or more lens, a filter, a optical beamsplitters, or any combination thereof. The optical configuration may further include a photodetector for detecting an optical signal from the immunoassay device. FIG. 9B depicts an example of an excitation/emission spectra with an excitation wavelength of 492 nm and an emission wavelength of 512 nm.

FIG. 10A depicts positioning of optical configuration at a position (e.g., Position A) of an immunoassay device. As a diagnostic test system of the disclosure moves longitudinally and scans across the test strip of the immunoassay device, an emission spectra is generated corresponding to increased optical signal at various regions along the test strip, as shown in FIG. 10B.

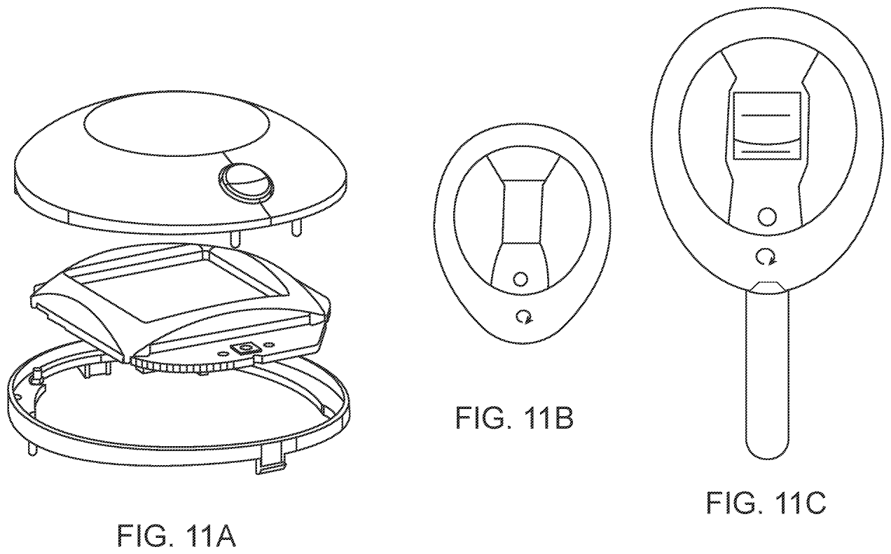

FIGS. 11A and B depict non-limiting examples of a diagnostic test system, demonstrating various design elements of the device. FIG. 11C depicts a non-limiting example of a top view of a diagnostic test system with an immunoassay test device inserted into the receiving chamber of the system.

In some cases, the diagnostic test device generates measurement results (e.g., concentration or relative amounts of analytes present in the sample) from a completed assay performed on the test device, as described throughout. In some cases, the diagnostic test device displays the measurement results on the device screen. Data containing the measurement results can be transmitted from the diagnostic test device to a mobile device and/or to a server. The data may be transmitted via one or more wireless or wired communication channels. The wireless communication channels may comprise Bluetooth®, WiFi, 3G, and/or 4G networks.

The data containing the measurement results may be stored in a memory on the diagnostic test device when the diagnostic test device is not in operable communication with the mobile device and/or the server. The data may be transmitted from the diagnostic test device to the mobile device and/or the server when operable communication between the diagnostic test device and the mobile device and/or the server is re-established.

A network can be configured to provide communication between the various components of the embodiments described herein. The network may be implemented, in some embodiments, as one or more networks that connect devices and/or components in the network layout for allowing communication between them. For example, one or more diagnostic test devices, mobile devices and/or servers may be in operable communication with one another over a network. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth®, Near Field Communication (NFC) technologies, networks based on mobile data protocols such as General Packet Radio Services (GPRS), GSM, Enhanced Data GSM Environment (EDGE), 3G, 4G, or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

One or more diagnostic test devices, mobile devices and/or servers may be connected or interconnected to one or more databases. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments.

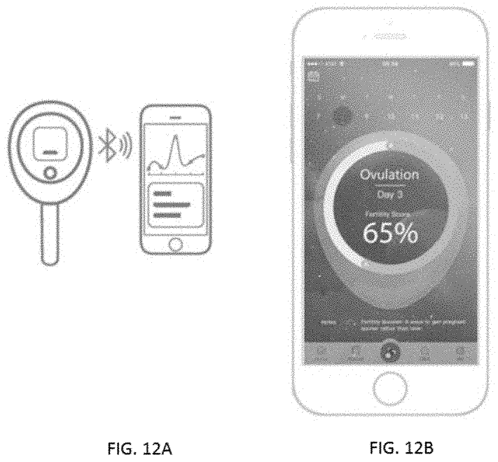

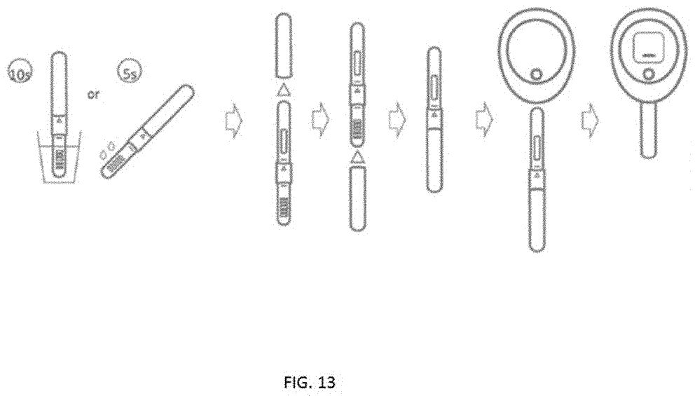

In some embodiments, one or more graphical user interfaces (GUIs) may be provided on the mobile device. The GUIs may be rendered on a display screen on the mobile device. A GUI is a type of interface that allows users to interact with electronic devices through graphical icons and visual indicators such as secondary notation, as opposed to text-based interfaces, typed command labels or text navigation. The actions in a GUI are usually performed through direct manipulation of the graphical elements. In addition to computers, GUIs can be found in hand-held devices such as MP3 players, portable media players, gaming devices and smaller household, office and industry equipment. The GUIs may be provided in a software, a software application, a web browser, etc. The GUIs may be displayed on the mobile device (e.g., FIG. 12B). The GUIs may be provided through a mobile application. The GUIs may be rendered through an application (e.g., via an application programming interface (API) executed on the mobile device). The GUIs may show images that permit a user to monitor fertility changes and levels.

As depicted in FIG. 12, the diagnostic test system may further comprise means for transmitting data generated by the diagnostic test system. In some cases, the data may be transmitted to and/or read from a mobile device (e.g., a cell phone, a tablet), a computer, a cloud application or any combination thereof. The data may be transmitted by any means for transmitting data, including, but not limited to, downloading the data from the system (e.g., USB, RS-232 serial, or other industry standard communications protocol) and wireless transmission (e.g., Bluetooth®, ANT+, NFC, or other similar industry standard). The information may be displayed as a report. The report may be displayed on the screen of a mobile device or a computer. The report may be transmitted to a healthcare provider or a caregiver. In some instances, the data may be downloaded to an electronic health record. Optionally, the data may comprise or be part of an electronic health record. For example, the data may be uploaded to an electronic health record of a user of the devices and methods described herein. In some cases, the data may be transmitted to a mobile device and displayed for a user on a mobile application, as shown in FIG. 12B.

Data collected by and transmitted by the diagnostic test system may include results of the immunoassay test performed on the immunoassay test device. For example, the data may include the concentrations of analytes (such as a first analyte and a second analyte) present in a sample. The concentration may relative concentrations or absolute concentrations. Data may also include an outcome such as a diagnostic outcome or a prognostic outcome, as shown in FIG. 12B.

Additional data that may be transmitted by the diagnostic test system include, without limitation, patient information, information obtained from the readable chip of the immunoassay device, time and date of the tests, system status (testing temperature, battery status, system self-testing and calibration results), error codes or error messages.

### Methods

Further provided herein are methods for using the immunoassay devices described herein and for performing diagnostic assays.

In one aspect, a method is provided for detecting the presence of a first analyte and a second analyte in a biological sample. The method may comprise: a) contacting a first end of a test strip with a biological sample suspected of containing the first analyte and the second analyte. The method may further comprise: b) reacting the biological sample at a labeling zone with a mobilizable first detection reagent conjugated to a first fluorescent label and a mobilizable second detection reagent conjugated to a second fluorescent label, which first detection reagent specifically binds to the first analyte thereby forming a first analyte-first detection reagent complex and the second detection reagent specifically binds to the second analyte thereby forming a second analyte-second detection reagent complex. The method may further comprise: c) flowing the biological sample from the labeling zone to a capture zone comprising a first capture region and a second capture region, wherein the first capture region comprises a first capture reagent immobilized thereon which specifically binds to the first detection reagent when the first detection reagent is not in a complex with the first analyte, and wherein the second capture region comprises a second capture reagent immobilized thereon which specifically binds to the second detection reagent-second analyte complex. The method may further comprise: d) detecting: (i) a first optical signal from the first fluorescent label present at the first capture region which first optical signal decreases with increasing amounts of the first analyte present in the biological sample; and (ii) a second optical signal from the second fluorescent label present at the second capture region which second optical signal increases with increasing amounts of the second analyte present in the biological sample, thereby detecting a presence of the first analyte and the second analyte present in the biological sample.

In various aspects, the methods may involve the use of one or more immunoassay test devices provided by the disclosure. The methods generally involve contacting the test device comprising a test strip with a sample by, for example, contacting a sample zone of the device with a fluid sample. Examples of samples suitable for use with the test devices have been described. The sample may be flowed along a flow path of the test strip from a proximal end to the distal end of the test strip. In some cases, the sample is flowed by capillarity or wicking.

FIG. 13 depicts an example workflow of a method of using an immunoassay test device to perform an immunoassay, and then obtaining a result using a diagnostic test system. An immunoassay test device is contacted with a fluid sample, either by inserting the test device into a container holding a fluid sample for a period of time (e.g., 10 seconds) or by holding the test device under a fluid stream for a period of time (e.g., 5 seconds). A cap may be removed from the distal end of the immunoassay device to reveal the assay results window and another cap may be inserted over the proximal end of the immunoassay device to cover and protect the sample zone. After waiting for a period of time for the immunoassay to be completed, the immunoassay test device may be inserted into the receiving chamber of a diagnostic test system, and the diagnostic test system may scan the immunoassay test device to obtain a result.

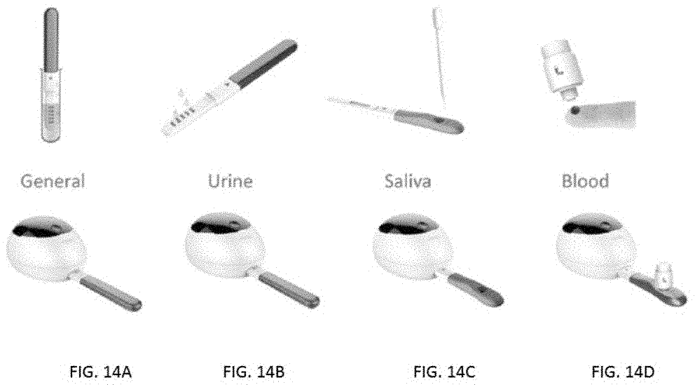

FIG. 14 depicts methods of applying various biological samples to a sample zone of an immunoassay device. Biological samples may include urine, saliva, and blood, among others as described herein. The fluid sample may be applied to a sample zone of an immunoassay test device by, e.g. inserting the proximal end of the device into a contained holding the sample, by holding the proximal end of the device under a fluid stream (e.g., a urine stream), by pipetting a fluid sample onto the sample zone of the device, or by using a needle or other pricking device to obtain a blood sample.

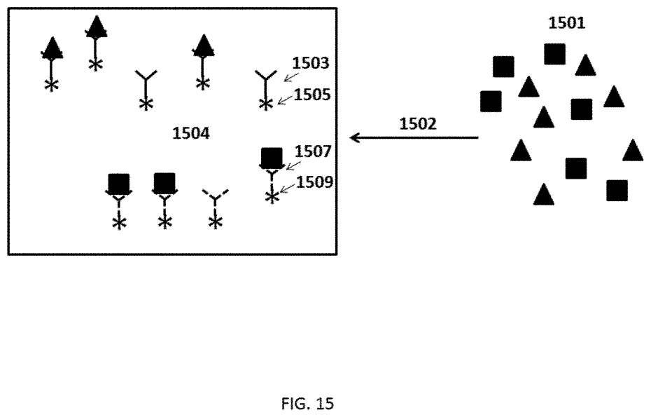

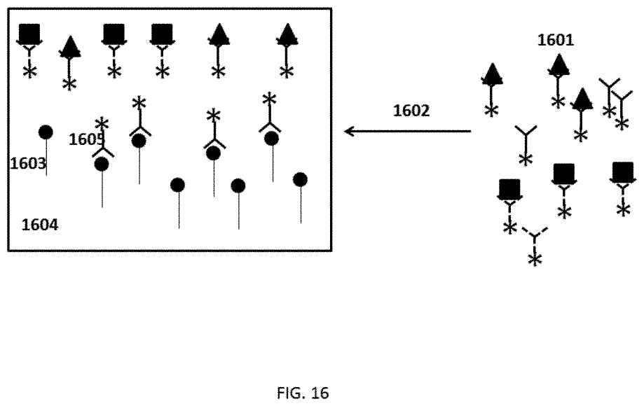

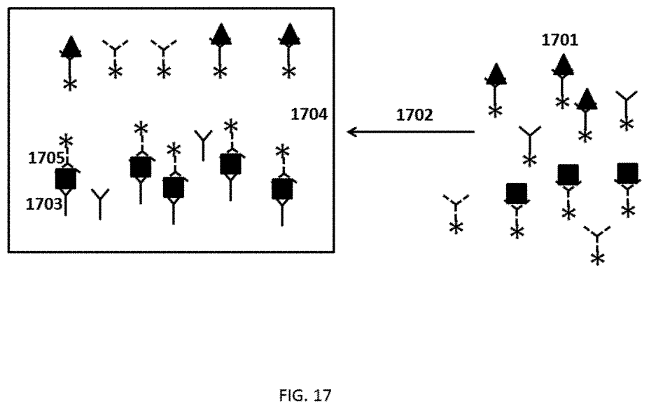

FIG. 15 depicts a non-limiting example of a labeling reaction as performed on a test device of the disclosure. A sample 1501 suspected of containing a first analyte (triangles) and a second analyte (squares) may be flowed 1502 from a sample zone to a labeling zone 1504 containing a mobilizable first detection reagent 1503 conjugated to a first detectable label 1505 and a mobilizable second detection reagent 1507 conjugated to a second detectable label 1509. The first detection reagent 1503 may be capable of selectively binding to the first analyte (triangles), when present in the sample, and the second detection reagent 1507 may be capable of selectively binding to the second analyte (squares), when present in the sample.

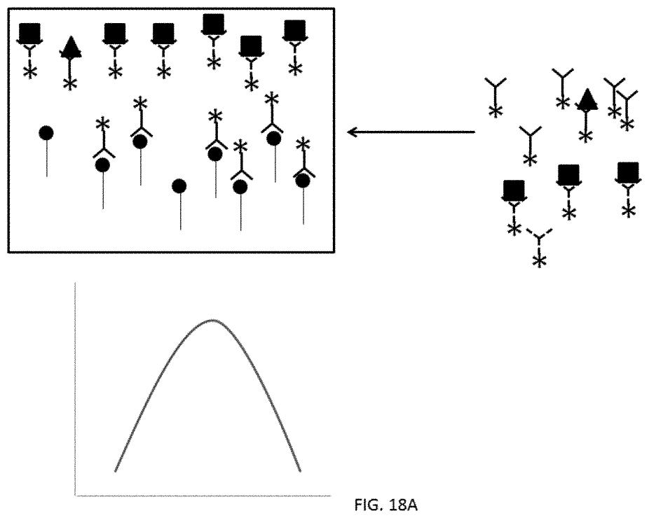

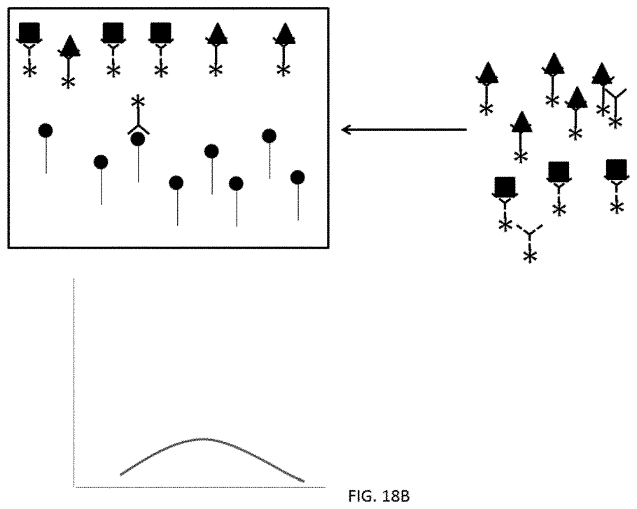

FIG. 16 depicts a non-limiting example of a competitive binding-based assay as performed on a test device of the disclosure. After a first analyte and second analyte, when present in the sample, have been labeled (see FIG. 15), the labeled sample 1601 may be flowed 1602 along the test strip from the labeling zone to a capture zone comprising a first capture region 1604. The first capture region 1604 may have immobilized thereon a first capture reagent 1603. The first capture reagent 1603 may be capable of specifically binding to non-complexed first detection reagent 1605 (i.e., when the first detection reagent is not in a complex with the first analyte). The first capture reagent 1603 may not be capable of binding to the first analyte or the first analyte-first detection reagent complex.

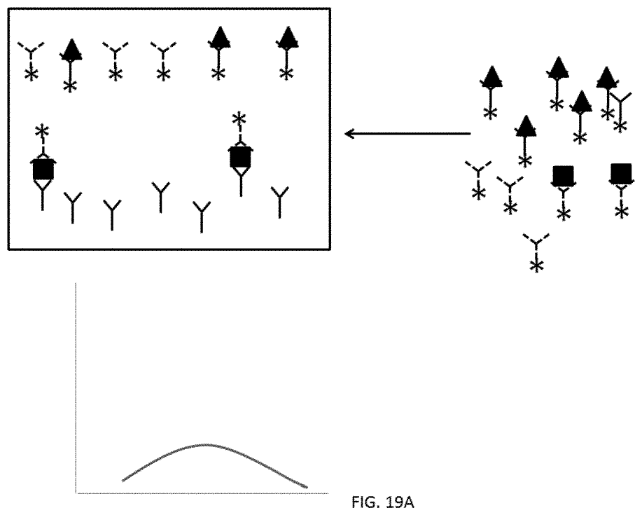

FIG. 17 depicts a non-limiting example of a sandwich-based assay as performed on a test device of the disclosure. After a first analyte and second analyte, when present in the sample, have been labeled (see FIG. 15), the labeled sample 1701 may be flowed 1702 along the test strip from the labeling zone to a capture zone comprising a second capture region 1704. The second capture region 1704 may have immobilized thereon a second capture reagent 1703. The second capture reagent may be capable of specifically binding to second analyte-second detection reagent complex 1705, for example, as formed by the labeling reaction in FIG. 15. The second capture reagent may not be capable of binding to the second analyte alone or the second detection reagent alone.

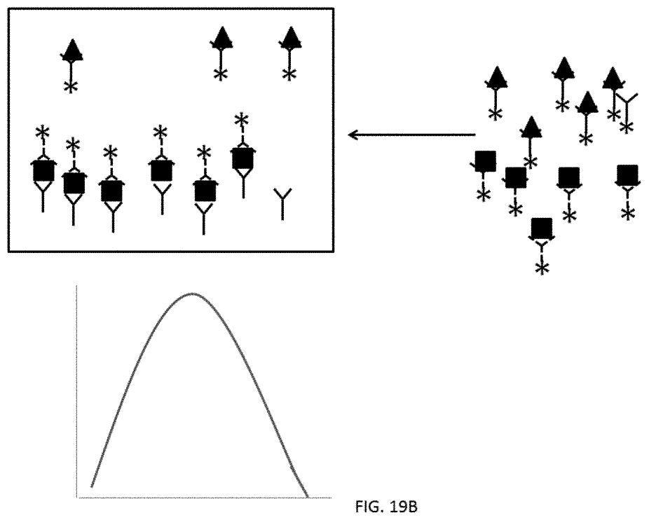

FIG. 18A depicts a non-limiting example of utilizing a competitive binding-based assay as performed on a test device of the disclosure to detect the absence of (or low levels of) a first analyte in the sample. A labeled sample (see FIG. 15) may be flowed from the labeling zone to a capture zone comprising a first capture region, as shown in FIG. 16. When the levels of first analyte in the sample are low, or absent, less first analyte-first detection reagent complex is formed at the labeling zone, and therefore, more free first detection reagent is available for binding to the first capture reagent immobilized at the first capture region. Thus, the signal produced by measuring the amount of first detectable label at the first capture region will be high. In contrast, FIG. 18B depicts a non-limiting example of utilizing a competitive binding-based assay as performed on a test device of the disclosure to detect high levels of first analyte present in the sample. When the levels of first analyte in the sample are high, more first analyte-first detection reagent complex is formed at the labeling zone, and therefore, less free first detection reagent is available for binding to the first capture reagent immobilized at the first capture region. Thus, the signal produced by measuring the amount of first detectable label at the first capture region will be low, or absent.

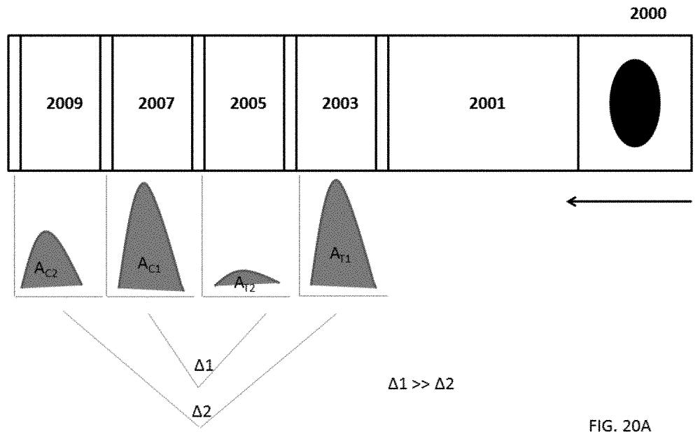

FIG. 19A depicts a non-limiting example of utilizing a sandwich-based assay as performed on a test device of the disclosure to detect the absence of (or low levels of) second analyte in the sample. A labeled sample (see FIG. 15) may be flowed from the labeling zone to a capture zone comprising a second capture region, as shown in FIG. 16. When the levels of second analyte in the sample are low, or absent, less second analyte-second detection reagent is formed at the labeling zone, and therefore, less second analyte-second detection reagent is available for binding to the second capture reagent immobilized at the second capture region. Thus, the signal produced by measuring the amount of second detectable label at the second capture region will be low. In contrast, FIG. 19B depicts a non-limiting example of utilizing a sandwich-based assay as performed on a test device of the disclosure to detect high levels of second analyte present in the sample. When the levels of second analyte in the sample are high, more second analyte-second detection reagent complex is formed at the labeling zone, and therefore, more second analyte-second detection reagent complex is available for binding to the second capture reagent immobilized at the second capture region. Thus, the signal produced by measuring the amount of second detectable label at the second capture region will be low, or absent.

### Data Analysis Methodology

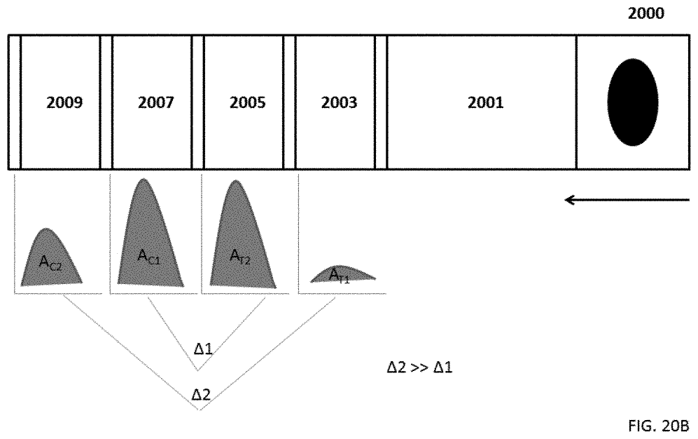

FIGS. 20A & 20B demonstrate the detection of a first analyte and a second analyte present in a sample by utilizing an immunoassay device of the disclosure. FIG. 20A demonstrates an example of a negative test result (e.g., when a first analyte and a second analyte are absent from or present in low amounts in a sample). The amount of first analyte present in the sample may be detected at the first capture region and the amount of second analyte present in the sample may be detected at the second capture region, in accordance with the disclosure. When the amount of first analyte present in the sample is low or absent, the signal produced at the first capture region is high, whereas when the amount of second analyte present in the sample is low or absent, the signal produced at the second capture region is low. Further, the sample may be further flowed to a control zone comprising a first control region and a second control region. The first control region and second control region have immobilized thereon a first and second control reagent, respectively, capable of binding to excess first and second detection reagent present in the control zone. As shown in FIG. 20A, optical signal intensity may be measured at each position along the immunoassay device. The area under the curve may be calculated to determine the signal intensity for each discrete region of the device. The difference between the signal intensity at the first control region (A_{C1}) and the signal intensity at the second capture region (A_{T2}) may be determined (Δ1). Further, the difference between the signal intensity at the second control region (A_{C2}) and the signal intensity at the first capture region (A_{T1}) may be determined (Δ2). Δ1 and Δ2 may be compared and a negative result is outputted if Δ1 is greater than Δ2. Similarly, FIG. 20B demonstrates an example of a positive result (e.g., both the first analyte and second analyte are present in the sample in high amounts). When the amount of first analyte present in the sample is high, the signal intensity at the first capture region is low, whereas when the amount of second analyte present in the sample is low, the signal intensity at the second capture region is high. The difference between the signal intensity at the first control region (A_{C1}) and the signal intensity at the second capture region (A_{T2}) may be determined (Δ1). Further, the difference between the signal intensity at the second control region (A_{C2}) and the signal intensity at the first capture region (A_{T1}) may be determined (Δ2). Δ1 and Δ2 may be compared and a positive result is outputted if Δ1 is less than Δ2.

In some cases, the difference between the signal intensity of the first capture region and the signal intensity of the second capture region may be calculated. In some cases, the difference between the signal intensity of the first control region and the signal intensity of the second control region may be calculated. In some cases, the difference between the signal intensity of the first test region and the first control region may be calculated. In some cases, the difference between the signal intensity of the second test region and the second control region may be calculated.

The methods and devices herein may be used to detect the presence of one or more analytes in a sample. The methods and devices herein may be capable of detecting at least about 5 ng/ml analyte in a sample. For example, the methods and devices herein may be capable of detecting at least about 5 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, 100 ng/ml, 150 ng/ml, 200 ng/ml, 250 ng/ml, 300 ng/ml, 350 ng/ml, 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, 1000 ng/ml or greater of analyte in a sample.

### Kits

Further provided herein are kits which may include any number of immunoassay test devices and/or reader devices of the disclosure. In one aspect, a kit is provided for determining qualitatively or quantitatively the presence of at least a first analyte and a second analyte in a biological sample, the kit comprising: a) an assay device according to an embodiment of the disclosure; and b) instructions for using the kit.

In some cases, kits may include a one or more immunoassay test devices of the disclosure. In some cases, the kit may provide a plurality of immunoassay devices to enable a user to conduct a test on more than one occasion. In some cases, the immunoassay devices are configured for a single use (i.e., are disposable). A kit may include a plurality of test devices to enable a user to perform a test once a day, once every 2 days, once every 3 days, once every 4 days, once every 5 days, once every 6 days, once every week, once every 2 weeks, once every 3 weeks, once every 4 weeks, once every 5 weeks, once every 6 weeks once every 7 weeks, once every 8 weeks or more.

In some cases, kits may include a plurality of immunoassay devices, each capable of detecting the same analytes. In other cases, kits may include a plurality of immunoassay devices, each capable of detecting different analytes. In a particular embodiment, a kit may include a plurality of immunoassay devices, each of the immunoassay devices capable of detecting the presence of E3G and LH in a biological sample. In another particular embodiment, a kit may include a plurality of immunoassay devices, each of the immunoassay devices capable of detecting the presence of LH in a biological sample. In another particular embodiment, a kit may include a plurality of immunoassay devices, each of the immunoassay devices capable of detecting the presence of hCG in a biological sample. In another particular embodiment, a kit may include a plurality of immunoassay devices, each of the immunoassay devices capable of detecting the presence of Influenza A & B in a biological sample. In another particular embodiment, a kit may include a plurality of immunoassay devices, each of the immunoassay devices capable of detecting the presence of 25-hydroxyvitamin D in a biological sample. In another particular embodiment, a kit may include a plurality of immunoassay devices, each of the immunoassay devices capable of detecting the presence of triiodothyronine (T3) and thyroxine (T4) in a biological sample. In another particular embodiment, a kit may include a plurality of immunoassay devices, each of the immunoassay devices capable of detecting the presence of one or more allergens in a food or drink product.

In some cases, kits can be provided with instructions. The instructions can be provided in the kit or they can be accessed electronically (e.g., on the World Wide Web). The instructions can provide information on how to use the devices and/or systems of the present disclosure. The instructions can provide information on how to perform the methods of the disclosure. In some cases, the kit can be purchased by a physician or health care provider for administration at a clinic or hospital. In other cases, the kit can be purchased by the subject and self-administered (e.g., at home). In some cases, the kit can be purchased by a laboratory.

Kits may further comprise a diagnostic test system of the disclosure. The diagnostic test system may be configured to be used with the immunoassay test devices of the disclosure. In some cases, the diagnostic test system is configured to be in operable communication with the assay device.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Methods of Detecting Levels of Estrone-3-Glucoronide and Luteinizing Hormone in a Urine Sample Obtained from a Female Mammal with a Device According to the Disclosure

Disclosed herein are methods and devices for detecting levels of estrone-3-glucoronide (E3G) and luteinizing hormone (LH) in a female mammal. In one non-limiting example, a device configured to detect the amounts of estrone-3-glucoronide (E3G) and luteinizing hormone (LH) present in a urine sample is provided herein. Without wishing to be bound by theory, increased levels of E3G and LH in a urine sample may indicate that a female mammal is at a time of peak or elevated fertility, and thus, an increased chance of becoming pregnant. On the other hand, low levels of E3G and LH in a urine sample may indicate that a female mammal is at a time of low fertility, and thus, a decreased chance of becoming pregnant. The methods and devices described herein may exhibit increased sensitivity as compared to competing devices currently on the market.

In one non-limiting example, a device and methods of using the device in accordance with embodiments described herein are provided. A female subject wishes to know if she is currently at a time of low fertility or high fertility. The subject takes a urine sample by urinating into a container and then dips a device of the disclosure into the urine sample such that the sample zone of the device is contacted with the urine sample. The urine sample is suspected of containing E3G and LH. The urine sample is flowed from the sample zone to a labeling zone. The labeling zone comprises a mobilizable anti-E3G antibody conjugated to a first fluorophore, and a mobilizable anti-LH antibody conjugated to a second fluorophore. The anti-E3G antibody is capable of binding to E3G, when present in the urine sample, thereby forming a complex with E3G. Similarly, the anti-LH antibody is capable of binding to LH, when present in the urine sample, thereby forming a complex a LH. The complexes, if formed, as well as any non-complexed anti-E3G antibody and anti-LH antibody are flowed from the labeling zone to the capture zone. The capture zone comprises two capture regions: a first capture region and a second capture region. The first capture region and second capture region may be positioned in any order on the flow path of the device, for example, the first capture region may be downstream from the second capture region, or the first capture region may be upstream from the second capture region. The first capture region comprises an immobilized protein-E3G antigen complex which is capable of binding to the anti-E3G antibody, but not E3G or a complex of E3G. When the levels of E3G present in the urine sample are low or absent, less E3G are available to bind to the anti-E3G antibody at the labeling zone, and more non-complexed anti-E3G antibody are available to bind to the immobilized protein-E3G antigen complex at the first capture region. In contrast, when the levels of E3G present in the urine sample are high, more E3G are available to bind to the anti-E3G antibody at the labeling zone, and less non-complexed anti-E3G antibody are available to bind to the immobilized protein-E3G antigen complex at the first capture zone. The second capture region comprises an immobilized anti-LH antibody which is capable of binding to complexed LH and anti-LH antibody formed at the labeling zone, but not non-complexed LH or non-complexed anti-LH antibody. When the levels of LH present in the urine sample are low or absent, less LH is available to bind to the anti-LH antibody at the labeling zone thereby forming less anti-LH antibody-LH complexes, and less anti-LH antibody-LH complexes are available to bind to the anti-LH antibody present at the second capture region. In contrast, when the levels of LH present in the urine sample are high, more LH is available to bind to the anti-LH antibody at the labeling zone thereby forming more anti-LH antibody-LH complexes, and more anti-LH antibody-LH complexes are be available to bind to the anti-LH antibody present at the second capture region.

In one non-limiting embodiment, the sample is further flowed from the capture zone to a control zone. The control zone comprises a first control region and a second control region. The first control region comprises an anti-mouse IgG capable of binding to any first and second detection reagents that are present in the first control region. Likewise, the second control region comprises an anti-mouse IgG capable of binding to any first and second detection reagents that are present in the second control region.

After the test has been conducted on the immunoassay device, the device is inserted into a diagnostic test device of the disclosure. The diagnostic test device is configured to scan the immunoassay device and measure the levels of fluorescent label present at the first capture region and the second capture region, corresponding to the amount of E3G and LH present in the original sample, respectively. The diagnostic test device is further configured to measure the levels of fluorescent label present at the first control region and the second control region corresponding to the amount of excess first detection reagent and second detection reagent present in the control zone.

The amounts of E3G and LH present in the urine sample are calculated by integrating to find the area under the curve of the signal intensity plots. The measurement results (e.g., amounts of E3G and LH present in the urine sample) may be displayed on a display screen of the diagnostic test device. The measurement results may further be transmitted to a mobile application on a mobile device (e.g., via Bluetooth®), as depicted in FIG. 12B.

The mobile application may perform one or more algorithms on the measurement results. For example, the difference between the signal intensity of the first capture region corresponding to the amount of E3G present in the sample and the signal intensity of the second control region is calculated (Δ1). The difference between the signal intensity of the second capture region corresponding to the amount of LH present in the sample and the signal intensity of the first control region is calculated (Δ2). Δ1 is compared to Δ2 and if Δ1 is greater than Δ2, a negative result is returned. In contrast, if Δ2 is greater than Δ1, a positive result is returned. In some cases, the algorithm may analyze the measurement results based on the user's historic data. For example, the algorithm may calculate one or more of the following: a) E3G and LH base line calculation; b) Previous cycle peak/maximum values and date; c) E3G increase slope.

A result may be displayed on a GUI provided on a mobile device for a user to view. The result may indicate the day of ovulation (e.g., a time from the last menstrual period) and/or may indicate a fertility score.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of detecting a presence of at least a first analyte and a second analyte in a biological sample, said method comprising:
a) contacting a first end of a test strip with a biological sample suspected of containing said first analyte and said second analyte;
b) reacting said biological sample at a labeling zone with a mobilizable first detection reagent conjugated to a first fluorescent label and a mobilizable second detection reagent conjugated to a second fluorescent label, which said first detection reagent specifically binds to said first analyte thereby forming a first analyte-first detection reagent complex and said second detection reagent specifically binds to said second analyte thereby forming a second analyte-second detection reagent complex;
c) flowing said biological sample from said labeling zone to a capture zone comprising a first capture region and a second capture region, wherein said first capture region comprises a first capture reagent immobilized thereon which specifically binds to said first detection reagent when said first detection reagent is not in a complex with said first analyte, and wherein said second capture region comprises a second capture reagent immobilized thereon which specifically binds to said second detection reagent-second analyte complex;
d) detecting: (i) a first optical signal from said first fluorescent label present at said first capture region which first optical signal decreases with increasing amounts of said first analyte present in said biological sample; and (ii) a second optical signal from said second fluorescent label present at said second capture region which second optical signal increases with increasing amounts of said second analyte present in said biological sample, thereby detecting a presence of said first analyte and said second analyte present in said biological sample.

2. The method of claim 1, wherein said first capture reagent does not displace said first analyte from said first detection reagent if said first analyte is initially bound to first detection reagent prior to step c).

3. The method of claim 1, wherein said first capture reagent does not bind to said first analyte-first detection reagent complex.

4. The method of claim 1, wherein said second capture reagent does not bind to non-complexed second analyte or non-complexed second detection reagent.

5. The method of claim 1, wherein said detecting of d) comprises: (A) illuminating said first capture region and said second capture region with one or more light sources; (B) detecting said first optical signal from said first capture region and said second optical signal from said second capture region with one or more optical detectors; (C) determining an amount of said first analyte present in said biological sample based on a level of said first optical signal; and (D) determining an amount of said second analyte present in said biological sample based on a level of said second optical signal.

6. The method of claim 1, wherein said first capture region is downstream of said second capture region on said test strip.

7. The method of claim 1, wherein said second capture region is downstream of said first capture region on said test strip.

8. The method of claim 1, wherein said first fluorescent label and said second fluorescent label are the same.

9. The method of claim 1, wherein said first analyte is estrone-3-glucuronide (E3G) and said second analyte is luteinizing hormone (LH).

10. The method of claim 9, wherein said first detection reagent comprises an anti-E3G antibody and said second detection reagent comprises an anti-LH antibody.

11. The method of claim 10, wherein said first capture reagent comprises a protein-E3G antigen complex and said second capture reagent comprises an anti-LH antibody.

12. The method of claim 11, wherein a decrease in said first optical signal and an increase in said second optical signal are indicative of a time of an elevated ovulation cycle of a mammal.

13. The method of claim 1, wherein said first fluorescent label and said second fluorescent label are different.

14. The method of claim 1, wherein said first fluorescent label and said second fluorescent label are the same.

15. The method of claim 1, wherein said first detection reagent, said second detection reagent, or both is an antibody or antibody fragment.

16. The method of claim 1, wherein said first detection reagent, said second detection reagent, or both is an antigen.

17. The method of claim 1, wherein said biological sample is blood, urine, or saliva.

18. The method of claim 1, wherein said first capture reagent is an antigen and said second capture reagent is an antibody.

19-26. (canceled)

27. An assay device for determining a presence of at least a first analyte and a second analyte in a biological sample, said assay device comprising:
a test strip defining a flow path and comprising:
a) at a first end, a sample zone configured to be contacted with a biological sample suspected of containing said first analyte and said second analyte;
b) a labeling zone having absorbed thereon a mobilizable first detection reagent conjugated to a first fluorescent label and a mobilizable second detection reagent conjugated to a second fluorescent label, which said first detection reagent specifically binds to said first analyte thereby forming a first analyte-first detection reagent complex and said second detection reagent specifically binds to said second analyte thereby forming a second analyte-second detection reagent complex;
c) a capture zone comprising a first capture region and a second capture region, wherein said first capture region has immobilized thereon a first capture reagent which specifically binds to said first detection reagent when said first detection reagent is not in a complex with said first analyte, and said second capture region has immobilized thereon a second capture reagent which specifically binds to said second analyte-second detection reagent complex, wherein a first optical signal from said first fluorescent label is capable of being detected at said first capture region and which said first optical signal decreases with increasing amounts of said first analyte present in said biological sample, and wherein a second optical signal from said second fluorescent label is capable of being detected at said second capture region and which said second optical signal increases with increasing amounts of said second analyte present in said biological sample.

28-46. (canceled)

47. A diagnostic test system, comprising:
a housing, comprising:
a) a port for receiving an assay device, said assay device comprising two or more capture regions;
b) a reader comprising:
i) one or more light sources for illuminating said two or more capture regions;
ii) one or more light detectors for detecting optical signals from said two or more capture regions; and
c) a data analyzer having one or more processors configured to: A) receive said optical signals; and B) determine an amount of at least a first analyte and a second analyte present in a biological sample based on said optical signals, wherein an optical signal of a first of said two or more capture regions increases with decreasing amounts of said first analyte present in said biological sample, and an optical signal of a second of said two or more capture regions increases with increasing amounts of said second analyte present in said biological sample.

48-60. (canceled)
